(19) United States
(12) Patent Application Publication
(10) Pub. No.: US 2026/0295140 A1
(43) Pub. Date: Oct. 1, 2026

(54) SYSTEM FOR FLUSHING A BODY CAVITY, A MEASUREMENT UNIT AND A SMART TUBING ASSEMBLY FOR THE SYSTEM

(71) Applicant: HAERMONICS BV, Eindhoven (NL)

(72) Inventors: Leonardus Jozef Lucas Maria KOCH, Eindhoven (NL); Wouter MARKUS, Eindhoven (NL)

(21) Appl. No.: 19/474,920

(22) PCT Filed: Apr. 12, 2024
(86) PCT No.: PCT/EP2024/059942

§ 371 Date: Oct. 13, 2025

(30) Foreign Application Priority Data
Apr. 13, 2023 (EP) 23167664.4

(51) Int. Cl. A61M 1/00

(52) U.S. Cl. CPC A61M 1/73; A61M 1/777; A61M 1/79; A61M 2205/3393; A61M 2205/7545; A61M 2209/084; A61M 2210/101; A61M 2210/122

(57) ABSTRACT

A flushing system for flushing a wound and/or a body cavity, in particular the pericardial cavity (PC) and/or the pleural cavity, of a patient is provided. The flushing system comprises a carrier unit configured to be moved, an inflow unit comprises at least one infusion fluid container containing infusion fluids, a collection unit configured to collect effusion fluids from the patient, wherein the carrier unit is configured to accommodate the inflow unit and the collection unit, wherein the inflow unit further comprises at least one first weighing unit for measuring a weight of the infusion fluids delivered to the patient, and wherein the carrier unit comprises a second weighing unit for measuring a weight of the effusion fluids collected in the collection unit.

### CROSS REFERENCE TO RELATED APPLICATIONS

The present application is a U.S. National Phase of International Application No. PCT/EP2024/059942 entitled “SYSTEM FOR FLUSHING A BODY CAVITY, A MEASUREMENT UNIT AND A SMART TUBING ASSEMBLY FOR THE SYSTEM”, and filed on Apr. 12, 2024. International Application No. PCT/EP2024/059942 claims priority to European Patent Application No. 23167664.4 filed on Apr. 13, 2023. The entire contents of each of the above-listed applications are hereby incorporated by reference for all purposes.

### TECHNICAL FIELD

The present invention relates to a flushing system and components for the flushing system. In particular, the present invention relates to a flushing system for flushing a body cavity (e.g. the pleural and/or the pericardial cavity) of a patient as a postoperative treatment in order to reduce the risk of cardiac tamponade or pulmonary edema, reduce postoperative blood loss and reduce the accumulation of blood and clots in the pericardial cavity and/or the pleural cavity. The flushing system incorporates unique features and components that improve the efficiency, accuracy, safety, and comfort of the flushing process.

### BACKGROUND AND SUMMARY

The flushing of body cavities is a common medical procedure that is employed for various therapeutic, postoperative and diagnostic purposes. Flushing systems are used, for example, for draining pleural or pericardial effusions, for irrigating the pleural space during thoracic surgery, for draining pericardial effusions or for administering medications or therapeutic agents.

Excessive postoperative blood loss which might amount to more than 21 per 24 hours or more than 200 ml per hour is a known cause of complications at cardiac surgery. Reoperation/re-exploration for bleeding is a strong independent risk factor for adverse outcome following cardiac surgery with higher mortality and morbidity rates. In addition, postoperative bleeding requiring multiple transfusions and surgical re-exploration is associated with higher costs, increased sternal wound infection, and transfusion-related infection.

To evacuate blood from the pericardial cavity, and if necessary, from the pleural cavities, chest tubes are left postoperative. However, when blood loss is excessive or when clots start to develop more rapidly, the drains often fail in their function to evacuate all accumulated blood. Stasis of clots and blood in the pericardial cavity and/or pleural cavity may lead to high fibrinolytic activity, maintenance of blood loss and in some cases to cardiac tamponade and, as a possible result, to excessive filling pressure of the heart.

Traditional flushing systems, however, often involve manual operation and may lack precision and uncertainty as to the quantity of fluids infused into the patient, leading to potential fluid imbalance, discomfort, and complications for patients.

It is therefore an object of the present invention to provide an improved flushing system configured to flush a wound and/or a body cavity, in particular the pericardial cavity and/or pleural cavity, of a patient. Advantageously, the flushing system incorporates distinctive features and smart components that enhances the efficiency, safety, and precision of the flushing system. Moreover, the utilization of the flushing system offers a straightforward and reliable system for controlling the flushing process in a precise manner.

This object is achieved by a flushing system having the features as described herein.

Accordingly, a flushing system for flushing flush a wound and/or a body cavity, in particular the pericardial cavity (PC) and/or the pleural cavity, of a patient, is provided. The flushing system comprises:
a carrier unit configured for mobility (configured to be moved),
an inflow unit comprises at least one infusion fluid container containing infusion fluids,
a collection unit configured to collect effusion fluids from the patient,
wherein the carrier unit is configured to accommodate the inflow unit and the collection unit,
wherein the inflow unit further comprises at least one first weighing unit for measuring a weight of the infusion fluids delivered to the patient, and
wherein the carrier unit comprises a second weighing unit for measuring a weight of the effusion fluids collected in the collection unit.

The invention is based on the basic idea to provide for a unique flushing system that is configured to accurately determine both the amount of the fluids infused into a patient and the amount of fluid which is removed from the patient body. In particular, the flushing system comprises two weight measuring units for measuring both the weight of the fluids infused into the patient, e.g. a body cavity or body cavities, and the weight of the fluids drained from the patient, e.g. into a collection unit. More particularly, the flushing system is configured to separately examine effusion fluids removed from various body cavities, such as the pleural cavity and the pericardial cavity. That is, the flushing system is configured to separately measure the effusion fluids removed from, e.g. the pericardial cavity and the pleural cavity, and preferably to report the same in real-time. Advantageously, the flushing system comprises two intermediate fluid containers, each configured to be in fluid communication with a body cavity, to thereby facilitate, e.g. via at least one sensor, a discrete analysis and measurements of the effusion fluids removed from that body cavity. The performance of the flushing system is further improved by the provision of novel upstanding filters (i.e. arranged in parallel to the direction of the fluid flow) within each of the intermediate fluid containers. This arrangement of the filters prevents the obstruction of fluid flow through the intermediate fluid containers due to blood clots caught in the filter. Additionally, the risk of aggregate formation, specifically the creation of larger blood clots, is effectively minimized. These aggregates might obstruct the flow of fluid in other components further downstream in the flushing system.

In particular, the flushing system further comprises a measurement unit that is configured to be operably connected to the inflow unit and the collection unit.

In particular, the measurement unit is in fluid communication with the inflow unit and the collection unit.

In particular, the measurement unit comprises at least one analysis chamber for a sensor unit, at least one processing/analysis unit and/or at least one pumping unit. In particular, the measurement unit is disposed in an intermediate position, along a longitudinal axis, between the inflow unit and the collection unit.

In particular, the measurement unit is configured to be arranged downstream from the inflow unit.

In particular, the measurement unit is configured to be arranged (within the carrier unit and) downstream from the inflow unit in the carrier unit.

In particular, the measurement unit comprises components, such as pump devices, sensors, valves, fluid containers, tubing, and/or tubing connectors that are assembled together and are configured, for example, for pumping the infusion fluids to the patient, for intermediate storage of the effusion fluids removed from the patient, and for analyzing the effusion fluids.

In particular, the measurement unit is configured to be connected to the collection unit of the flushing system (i.e. a vacuum line thereof), allowing to discharge the removed infusion fluids to, e.g. a waste container.

In particular, the measurement unit comprises two intermediate fluid containers (e.g. buffer containers) for receiving (or containing) the effusion fluids, preferably a first intermediate fluid container configured to receive the effusion fluids from a pleural cavity, and a second intermediate fluid container for receiving the effusion fluids from the pericardial cavity.

In particular, the flushing system may further comprise one or more suction devices to draw effusion liquid out of the wound and/or the body cavity, in particular the pericardial cavity. The one or more suction devices may be arranged to create a relative low pressure in the one or more effusion liquid containers to receive effusion liquid so that effusion liquid is drawn towards the one or more effusion liquid containers. For example, the one or more effusion liquid containers may be connectable or connected to an intensive care unit (ICU) vacuum wall connector or the like (e.g., any other suitable vacuum providing unit/system and/or any other suitable custom made vacuum unit), wherein the ICU vacuum wall connector may be configured to create said (relative) low pressure and may thereby act as said one or more suction devices. Additionally or alternatively, a system's own vacuum unit may also be provided, in particular for the above-described purpose.

In particular, each of the buffer containers may substantially have a rectangular shape extending along a central or longitudinal axis. However, it should be understood that any other shape, such as a cylindrical or oval shape, may be possible in this regard. The buffer container may comprise a top end and a bottom end. The buffer container may comprise a top wall, a side wall and a bottom wall. The buffer inlet may be disposed closer to the top wall than to the bottom wall. The first buffer outlet may be disposed closer to the bottom wall than to the top wall. The buffer inlet may be arranged in the top wall or the side wall. The first buffer outlet may be arranged in the bottom wall or the side wall. Hence, the buffer inlet may be arranged at a higher level than the first buffer outlet with respect to the gravitational direction. When the buffer container is incorporated, e.g., mounted, in the system, the central axis substantially extends along the gravitational direction.

In particular, each of the buffer containers may further comprise a second outlet (a second buffer outlet) being arranged at a higher level than the first buffer outlet with respect to the gravitational direction and being configured to output effusion liquid and/or air, to thereby allow an air-liquid-separation and/or allow to function as the safety bypass. Further, the second buffer outlet may be arranged at a lower level than the buffer inlet with respect to the gravitational direction. The second buffer outlet may be connectable or connected to the one or more effusion liquid containers. For example, the second buffer outlet may be connectable or connected to the one or more effusion liquid containers in a direct and/or an always open manner.

The second buffer outlet may be disposed on an opposite side of the clot trap with respect to the first buffer outlet. Hence, the second buffer outlet may be disposed on the same side as the buffer inlet.

The second buffer outlet may act as a safety overflow and/or a safety bypass from the buffer container, to thereby provide an open connection to the one or more effusion liquid containers for safety reasons (e.g., for preventing back pressure/afflux). Further, the second buffer outlet may also provide an exit path for air being separated from the effusion liquid in the buffer container. The separated air may be sucked into the one or more suction devices, e.g., the ICU vacuum wall connector and/or a system's own vacuum unit. In other words, the separated air may be sucked via the one or more effusion liquid containers into the ICU vacuum wall connector and/or into the system's own vacuum unit. Hence, due to the lack of air in the effusion liquid, the effusion liquid may subsequently be analyzable or analyzed in a more accurate and reliable manner.

The flushing system may be configured to provide approximately 500 ml of infusion liquid per hour, i.e., the infusion inflow may be approximately 500 ml per hour. Hence, the buffer volume may be sized accordingly. However, the system and, accordingly, the buffer volume may be configured to handle sudden gushes of 400 ml and/or 500 ml of effusion liquid per 5 min.

In particular, the flushing system further comprises a tubing unit configured to be connected to the carrier unit, wherein the tubing unit is configured to carry the infusion fluids from the carrier unit towards the patient, and to carry the effusion fluids from the patient (e.g. the pericardial cavity and/or pleural cavity) towards the carrier unit.

In particular, the tubing unit is configured to be connected to the measurement unit.

In particular, the tubing unit comprises a plurality of inflow tubes for carrying the infusion fluids, and a plurality of outflow tubes for removing the effusion fluids from the patient, e.g. from the pericardial cavity and pleural cavity as appropriate.

In particular, the flushing system further comprises a user interaction unit configured to allow a user to monitor flushing processes and/or physiological conditions of the patient.

In particular, the user interface unit is configured to report results of analysis, that is individually conducted via the at least one sensor, for the effusion fluids removed from the pericardial cavity and the pleural cavity.

In particular, the user interface unit is configured to be coupled onto the carrier unit, preferably on an outer periphery of at an uppermost position of the carrier unit (along a longitudinal axis).

Alternatively, the user interface unit is in the form of a portable user interface unit which is configured to be connected to the flushing system via a wired or wireless connection.

In particular, the flushing system further comprises a base unit configured to facilitate the movement and transportability of the carrier unit. For example, the base unit is configured to be attached to the carrier unit at the lowermost position (along a longitudinal axis).

In particular, the base unit comprises a frame plate (a footplate) attached to the carrier unit, wherein the frame plate configured to form (or create) a rim or an outer edged around the periphery of the carrier unit.

The base unit further comprises driving elements, such as wheels, enabling the carrier unit to move over the ground.

In particular, the inflow unit comprises at least one mounting element for holding at least part of the at least one first weighing unit and/or the at least one infusion fluid container in the inflow unit.

In particular, the at least one first weighing unit is configured to retain the at least one infusion fluid container.

In particular, the infusion fluid container may comprise the standard saline bags (e.g. 1 L, 2 L, 3 L or any combination thereof up to 5 L.

In particular, the at least one first weighing unit is configured to be connected to the carrier unit via a shock mounting.

In particular, the flushing system, e.g. the carrier unit, comprises at least one sensor unit for analyzing the effusion fluids collected in the measurement unit (e.g. in an analysis chamber) and/or a second pumping unit (an outflow pumping unit) for discharging the effusion fluids from the measurement unit.

In particular, the flushing system further comprises a control unit that is configured to generate control signals for regulating the infusion fluid flow rates of the first pumping unit to control and/or for controlling the suction rates of the effusion fluids of the second pumping unit, for example, in response to the measured fluid level in the intermediate fluid containers.

In particular, the at least one weighing unit or the control unit are be configured to determine the flow rate of infusion liquid pumped out of the infusion liquid container. It is remarked that any other sensor configured to determine the flow rate of infusion liquid towards the pericardial cavity PC may be used. For example, the flow rate of infusion liquid may be determined by a flow rate sensor which may be located anywhere in the inflow unit of the flushing system. In particular, the flow rate of infusion fluid may also be deducted from a control signal provided by the control unit to adjust the flow rate of the first pumping unit in response to one or more of the sensor signals.

In particular, the carrier unit comprises a housing extending along a longitudinal axis from a bottom position to a top position.

In particular, the housing is configured to house the collection unit, the measurement unit, and the inflow unit therein, respectively, from the bottom position to the top position. For example, the collection unit, the measurement unit and the inflow unit are configured to be stacked in the housing along the longitudinal axis.

In particular, the collection unit is in the form of a drawer element that is configured to be removably arranged in the carrier unit. For example, the collection unit is configured to slide into a corresponding compartment provided in the housing of the carrier unit at a bottom position thereof.

In particular, the second weighing unit is configured to be disposed (integrated) in the carrier unit, e.g. in the compartment that receives the collection unit.

In particular, the base unit is configured to be attached to the housing at the bottom position thereof.

In particular, the user interface unit is configured to be attached to the housing, e.g. at the top position or on one side of the housing (at the periphery of the housing).

In particular, the housing comprises a pass-through chamber (a hollow chamber) configured to accommodate the inflow unit therein. For example, the hollow chamber has two open sidewalls.

In particular, the inflow unit further comprises at least one inflow tube configured to carry (or convey) the infusion fluid from the infusion fluid container.

In particular, the inflow unit is in fluid communication with the measurement unit.

In particular, the housing of the carrier unit comprises a peripheral wall, extending from the base (bottom) portion to the top portion along a longitudinal axis.

In particular, the user interface unit is configured to be attached onto the housing, preferably on an outer periphery of the housing in the top position.

In particular, the carrier unit further comprises an inner frame configured to be disposed (or housed) in the housing of the carrier unit.

In particular, the inflow unit, the measurement unit and the collection unit are configured to be attached to the inner frame of the housing.

For example, the inflow unit, the measurement unit and the collection unit are configured to be removably disposed in the inner frame.

In particular, the at least one mounting element is configured to be connected to the inner frame, preferably on an inner wall of the hollow chamber.

In particular, the at least one first weighing unit comprises a case for retaining (or enclosing) the infusion fluid container. For example, the case is in the shape of an inverted U-form or a hollow cup.

In particular, the at least one mounting element comprises at least one mechanical fastener or snapping element configured to hold (or catch) the case or the infusion fluid container, for example, at least one hook.

In particular, the at least one first weighing unit is configured to be connected to the inner frame by way of a shock mounting mechanism.

In particular, the at least one first weighing unit (e.g. containing the infusion fluid container) is configured to be attached to the inner frame in a floating arrangement, being mechanically decoupled from the carrier unit.

In this way the first weighing unit is mounted in the carrier unit but it is mechanically decoupled (or isolated) from the carrier unit (and its components). This in turn, results in accurate and reliable weight measurements independent of external forces applied to the carrier unit.

In particular, the measurement unit comprises a mount plate, wherein the buffer containers are configured to be mounted on the mount plate.

In particular, the mount plate comprising an upper edge and a lower edge, which edges define a mounting surface therebetween. The measurement is configured to guide the effusion fluids from the upper edge to the lower edge thereof.

In particular, the mount plate includes an entrance opening formed at the upper edge of thereof. The entrance opening is configured to receive a first pumping unit (an inflow pump unit) for pumping the infusion fluid received from the inflow unit towards the patient.

In particular, the first pumping unit comprises a plurality of pumping devices configured to provide a plurality of inflow lines for the infusion fluid.

In particular, each of the pumping devices is configured to pump the infusion fluids received from the infusion fluid container via the tubing unit to the patient.

For example, the first pumping unit comprises three pumping devices that are stacked along a common axis (e.g. a travers axis perpendicular to the longitudinal axis).

In particular, each pumping device of the first pumping unit is configured to be connected to an inflow tube carrying the infusion fluid, and to an outflow tube pumping the infusion fluid toward the patient, e.g. to the pleural cavity and/or the pericardial cavity.

In particular, the outflow tubes of the pumping devices of the first pumping unit are in fluid communication with the tubing unit, in particular in fluid communication with the plurality of the inflow tubes included in the tubing unit.

In particular, the measurement unit further is configured to be in fluid communication with a second pumping unit (an outflow pumping unit), via connector tubes. The second pumping unit is configured to remove the effusion fluids from the patient, preferably from the pericardial cavity and/or pleural cavity.

In particular, the connector tubes are arranged downstream of the buffer containers along the direction of the effusion fluid flow. In other words, the buffer containers are disposed on the mount plate close to the upper edge thereof and the connector tubes close to the lower edge of the mount plate.

In particular, the second pumping unit comprises two outflow pumping devices, each configured to remove the effusion fluids, through the butter containers, from the pericardial cavity or the pleural cavity.

In particular, the buffer containers of the measurement unit, each are configured to receive, via the tubing unit, the effusion fluids from the pericardial cavity or pleural cavity.

In particular, each of the buffer containers comprises an inlet in fluid communication with one outflow tube of the plurality outflow tubes of the tubing unit that is connected to the patient.

Each of the buffer containers further comprises an outlet in fluid communication with one of the outflow pumping devices of the second pumping unit.

In particular, each of the buffer containers comprises a filter (or a clot trap) disposed within the buffer containers, preferably along the direction of the flow of the effusion fluids. For example, the clot trap is disposed vertically in parallel to the longitudinal axis of the carrier unit.

This arrangement of the filter with respect to the direction of the flow mitigate the blockage of the filter and provide for an improved flow for the removed effusion fluid through the flushing system.

In particular, the clot trap is configured to filter particles or components in the effusion fluid, for example, with a diameter ranging from 1 to 5 mm, preferably 1.5 to 3 mm.

In particular, each of the filters (the clot traps) comprise a sieve structure being disposed between the inlet of the intermediate container and the first outlet of the intermediate container (i.e. between the buffer inlet and the first buffer outlet of the buffer container), to thereby prevent clots from passing through the first buffer outlet. The clot trap may be integrally formed with the buffer container (e.g., on the side wall or the bottom wall of the buffer container). Alternatively, the clot trap may be mounted to the buffer container via fixing means. The sieve structure may comprise a plurality of openings formed therein. The openings may be configured to stop clots from passing through the sieve structure.

The clot trap may have a conical shape having a base facing towards the first buffer outlet and an apex facing the buffer inlet. It is also possible, that the clot trap may have other shapes, e.g., a rectangular shape, a square shape, a truncated cone shape, a dome shape, a flat shape etc. In particular, due to said shape of the clot trap and the arrangement with the buffer container, certain amounts of clots may be trapped in the clot trap without blocking the clot trap.

In this way, the blood clots are prevented from passing through the remaining parts of the flushing system. This in turn, prevents the formation of the larger blood clots in those parts of the flushing system, e.g. in the sensor or the second pumping device.

In particular, the second pumping unit is in fluid communication with an analysis chamber (a cuvette) of a sensor unit for analyzing the effusion fluid received from the buffer containers.

For example, a connector element (e.g. a Y-connector) is provided that is in fluid communication with outputs of outflow pumping devices of the second pumping unit.

In particular, the connector element is configured to guide the effusion fluids received from the intermediate fluid containers to the analysis chamber of the sensor unit, e.g. a hematocrit sensor, wherein the outflow pumping devices of the second pumping units are configured to operate alternately.

In this way, the effusion fluids received from the pericardial cavity or from the pleural cavity can be analyzed separately.

In particular, a hematocrit sensor is configured to measure the hematocrit value of the effusion liquid for obtaining a blood loss volume and/or a blood loss flow rate from the wound and/or the body cavity, in particular the pericardial cavity. The hematocrit sensor may be configured to optically measure said hematocrit value based on scattering and absorption of light emitted into the effusion liquid.

By a hematocrit analysis and/or measurement of the outflow fluids, i.e., effusion liquid, the exact amount of blood loss may be calculated more accurately. This may improve clinical decision making with regard to optimal timing of re-exploration and blood transfusion policy.

The hematocrit sensor may be disposed for conducting measurements in the effusion liquid pumped from the buffer container to the effusion liquid containers. Hence, the hematocrit sensor may be arranged downstream from the intermediate buffer container and upstream from the one or more effusion containers. Due to said arrangement of the hematocrit sensor, precise hematocrit measurements may be conducted without negative effects of clots and/or air in the effusion liquid. Further, since the second pumping device may be controlled with respect to the volume of liquid to be pumped by the control unit, the measured hematocrit value may be set in correlation with the pumped volume, in which the hematocrit value is measured by said sensor. Hence, the blood loss volume and/or the blood loss flow rate from the wound and/or the body cavity, in particular the pericardial cavity, may be measured in an easy, reliable and/or accurate manner.

It may also be conceivable, that the system may further comprises a unit for blood loss measurements and/or patient systematic measurements, which is configured for obtaining the blood loss volume and/or the blood loss flow rate from the wound and/or the body cavity, in particular the pericardial cavity (PC), and/or may be connectible to such a unit.

For example, the measurement unit comprises a support element including a plurality of fixing elements such as mounting clamps or clips for mounting the components of the measurement unit thereon.

Adventurously, the mount plate serves the function of a support element for supporting and fixing a position of e.g. the first pumping unit, the analysis chamber (cuvette), and the connector element included in the measurement unit.

In particular, the measurement unit further comprises two safety elements (e.g. overflow exits/tubes) disposed on the mount plate. Each of the safety elements are in fluid communication with one of the buffer containers, e.g. via a second buffer outlet.

The safety elements, each are configured to discharge the effusion fluids from the corresponding buffer containers.

In particular, the safety elements comprise safety overflow outlets (a second outlet of the intermediate fluid container) being in fluid communication with the collection unit, e.g. with a collection container included in the collection unit.

Advantageously, the safety elements serve the purpose of ensuring a continuous open airway for the vacuum to pass from the pump (pumping unit) in the flushing system to the patient. This is necessary as the vacuum cannot pass through, for example, peristaltic pumps. Additionally, the safety elements provide an overflow as a safety measure in case of a malfunction.

In particular, the measurement unit further comprises a discharge tube being in fluid communication with the collection unit.

In particular, the discharge tube is fluidly connected to the at least one sensor and the collection unit and is configured to guide the effusion fluid to the collection unit.

In particular, the measurement unit further comprises an aperture (e.g. formed on the mount plate) to retain a pressure line or air tube therethrough.

In particular, the flushing system further comprises a gripping element that is connected to the carrier unit. Preferably, the gripping element is arranged on the peripheral wall of the carrier unit at a position to provide a barrier in front of the hollow chamber of the carrier unit and to provide a grip (a handlebar/handgrip) for moving the carrier unit.

In this way, the carrier unit can be held, moved, pushed or pulled by the user as desired, while protecting the at least one first weighing element from accidental damages.

In particular, the gripping element comprises an arm element (e.g. a rod or bar structure) that is connected to the housing and at least partly extends around a periphery of the housing, thereby providing a barrier at one side of the hollow chamber.

In particular, the flushing system further comprises a locking element that is connected to the carrier unit, preferably the locking element is connected to or it is an integral part of in the inner frame (disposed on the housing).

In particular, the locking element is configured to fix a position of the at least one inflow tube exiting the inflow unit from moving. This ensures that the inflow tubes remain securely in one specific (defined) place, preventing any adverse impact on the measurement and analysis caused by variations in tube positioning or its placement in the carrier unit.

In particular, the tubing unit comprises a first tubing section connected to the flushing system, a third tubing section connected to the patient and a second tubing section disposed between the first and second tubing sections.

In particular, the third tubing section comprises a tubing assembly including an assembly of inflow tubes and at least one outflow tube, forming a multi-lumen tubing set that is configured to be in fluid communication with the patient.

Advantageously, by way of providing a bundle of tubes joined together as one single tubing assembly, handling and organizing the inflow and outflow tubes connected to the patient is improved.

In particular, the tubing unit comprises two tubing assemblies configured to be fluid connected to (be placed into) the pericardial cavity and the pleural cavity, respectively.

In particular, the tubing unit in the third section thereof may further comprise two further outflow tubes (e.g. two distinct (separate) outflow tubes that are not part of the tubing assembly). Each of the individual outflow tubes is configured to be fluidly connected to the pericardial cavity or the pleural cavity as appropriate.

In particular, the plurality of inflow tubes included in the tubing unit comprises, for example, three inflow tubes carrying the infusion fluid, preferably the three inflow tubes extending along the first, second and third sections of the tubing unit.

In particular, the tubing unit further comprises a pressure measurement tube for carrying air (an air tube), preferably extending along the first, second and third tubing sections.

In particular, the pressure measurement tube is configured to support, via the air channel in the tube, pressure measurement by a sensor within the system to the point of measurement, e.g. in the pericardial cavity of the patient.

The purpose of pressure measurement is to enable clinicians to monitor pressure levels and detect any trends. For example, a positive trend, i.e. increasing of pressure, may indicate an obstructed drain, necessitating inspection and potentially correction by the clinician.

In particular, the pressure measurement tube comprises an air tube that is, for example, disposed in or retained by the measurement unit and the tubing unit. For example, an air channel that extends from the measurement unit through the tubing unit into the patient's body.

In particular, the pressure measurement tube is configured to be connected on one end to the flushing system (e.g. via a connector such as Luer lock connector, to a pressure sensor) and on the other end to a tubing assembly (e.g. smart tube) placed in the patient.

In particular, the collection unit further comprises a waste container for collecting the effusion fluids from the patient, and a cover element that is removably connected to the waste container.

In particular, the collection unit further comprises at least one filter element for preventing biological contaminations from entering the environment.

Further, a first coupling element is provided in the collection unit for connecting the collecting unit with a vacuum pump (e.g. disposed in the carrier unit), and at least one second coupling element for connecting the collecting unit to the measurement unit, preferably to the discharge tube.

In particular, the collection unit is removably disposed within the carrier unit, preferably the collection unit comprises slide elements for sliding the collection unit into the housing (or the inner frame) of the carrier unit.

In particular, the waste container is configured to collect about 4 or 5 L of effusion fluid.

In particular, the collection unit further comprises a handle for carrying the collection unit, preferably the handle is hinged to/pivotally attached to the waste container and/or the cover element of the collection unit.

In particular, the filter element, the first coupling element and the second coupling element are disposed in the cover element.

In particular, the collection unit further comprises an overflow protection valve for protecting the first coupling element against fluid overfill.

In particular, the flushing system further comprises guiding elements configured to guide positioning of the tubing unit towards the carrier unit.

In other words, the guiding elements are arranged and configured to guide the alignment of the tubing unit in a manner where the tubing unit's position along a longitudinal axis descends in elevation from the patient towards the carrier unit. This promotes the flow of the effusion fluids towards the carrier unit.

In particular, the guiding elements are configured to guide a first section of the tubing unit (that is connected to the measurement unit) so that the flow of the effusion fluids occurs in the direction of gravity. By lowering the position of the tubing unit in height (along a longitudinal axis) from the patient towards the carrier unit, fluid drainage from the body cavity is further improved.

In particular, the guiding elements are connected to the carrier unit, preferably the guiding elements are connected to/disposed onto the peripheral wall (an outer wall) of the housing of the carrier unit.

In particular, the guiding elements comprise a plurality of tabs or retaining elements that are disposed on the outer wall of the housing and that are configured to direct, at least in part, the first section of the tubing unit on the housing.

In particular, the guiding elements (the plurality of tabs) is configured to guide the placement of the tubing unit on the housing so that the tubing unit's position along a longitudinal axis progressively lowers from the patient side toward the carrier unit e.g. the entrance opening of the measurement unit.

In particular, the flushing system further comprises a tubing interface unit configured to hold, at least partly, the tubing unit in place, wherein the tubing unit is configured to provide a fluid communication between the flushing system (e.g. the carrier unit) and the patient.

In particular, the flushing system further comprises at least one tubing connector that is provided in the third section of the tubing unit.

In particular, the at least one tubing connector is configured to combine the at least one outflow tube included in the tubing assembly with one of the separate (distinct) outflow tube.

In other words, the at least one tubing connector is included in the third section of the tubing unit, and is configured to combine outflow tubes connected to a specific body cavity, e.g. the pleural cavity or the pericardial cavity.

For example, the flushing assembly may comprise two fluid connectors connected to the corresponding tubing assemblies that are connected to the pericardial cavity and the pleural cavity, respectively.

For example, in cases where, further distinct outflow tubes for the pericardial cavity and plural cavity are utilized, theses outflow tubes are configured to be merged, via two tubing connectors, with the corresponding outflow tubes of the tubing assembly to thereby create one single outflow tube for each of the pericardial cavity and pleural cavity in the second and first tubing section of the tubing unit.

In particular, the flushing system further comprises a heater unit configured to heat, for example, a section of the tubing unit that is retained by the tubing interface unit.

In particular, the tubing interface unit is configured to be connected to a heater unit, wherein the plurality of inflow tubes included in the tubing unit is configured to be heated via the heater unit.

In particular, the heater unit is configured to heat the infusion liquid that is intended to be guided to the wound and/or the body cavity, in particular the pericardial cavity, to a desired infusion liquid temperature.

The heater unit, for example, comprises heating elements.

In particular, the heater unit may be part of a temperature control system of the flushing system and is configured to control a temperature of the infusion liquid.

In particular, the temperature control system comprises a sensor to measure a temperature of the heating elements and a temperature control unit for controlling the temperature of the heating elements.

In particular, the heater unit (or the heating elements) is configured (designed and arranged) to comprise (provide) a contact surface that is configured to get heated by the heating elements and transfer the heat to the inflow tubes passing through the tubing interface unit ereon.

In particular, the control unit is further configured to estimate the temperature of the fluid passing over the contact based on the interaction of the infusion fluids and the contact surface, e.g. contact time, dimensions of contact surface and fluid flow rate (the flow rate of the fluid that streams along the surface).

In particular, the heater unit is configured to be controlled/adjusted by the temperature control signal to heat the heating elements to a desired temperature. The temperature sensor may be arranged in or on the inflow tubes and/or outflow tube.

Alternatively, the temperature control system may comprise a temperature sensor to measure, for example, a temperature of infusion liquid, a temperature control unit, which may be part of the control unit or a separate unit of the system and which may be configured to provide a temperature control signal on the basis of the measured temperature of infusion liquid and a desired infusion liquid temperature, and said heater unit, wherein the heater unit may be controlled/adjusted by the temperature control signal to heat the infusion liquid to the desired infusion liquid temperature. The temperature sensor may be arranged in or on the inflow tubes and/or outflow tube.

In particular, the tubing interface unit comprises a casing including a base plate and a (peripheral or outer) wall extending upwardly from the base plate, thereby defining a chamber for accommodating at least partly the tubing unit.

For example, the casing includes a plurality of openings formed on one side wall and another (opposite) side wall of the casing.

In particular, the tubing interface unit is configured to direct, via the plurality of the openings, the plurality of the inflow tubes and/or the plurality of outflow tubes from one side wall through the chamber to the other side wall of the casing.

In this way, the tubing unit are assembled together, enabling better management and organization of the tubing unit. This in turn provides for an improved securing and positioning of the tubing unit with respect to the patient and the carrier unit.

In particular, the base plate/structure is configured to be connected to and to get heated via the heater.

In particular, the tubing interface unit further comprises an inner structure (disposed within the casing) defining a network of passages (e.g. meander paths or curved paths) for each of the inflow tubes of the plurality of inflow tubes to pass therethrough.

In this way, each of the inflow tubes follows an extended path through the casing of the tubing interface unit. Consequently, this prolongs the contact time with the heater unit, effectively warming the infusion fluid to a desired temperature, such as body temperature.

In particular, the tubing interface unit is configured to hold the second section of the tubing unit.

In particular, the tubing interface unit is in the form of a hub that is configured to retain the tubing unit and to facilitate heating of the plurality of inflow tubes carrying the infusion fluid.

In particular, the tubing interface unit further comprises a lid that is configured to be placed onto the casing for closing the same.

In particular, the base plate of the casing comprises a conductive (e.g. metallic material) for enhancing heat transfer to the partitioning elements.

For example, the partitioning elements can be designed in the form of a labyrinth.

In particular, the tubing interface unit comprises a first end, for example, for receiving the plurality of inflow tubes from the flushing system (e.g. system end/side), and a second end, for example, for receiving the plurality of outflow tubes from the patient (e.g. patient end/system side).

In particular, the flushing system further comprises at least one tubing connector, for example, for combing two outflow tubes of the tubing unit to one outflow tube.

For example, two of the outflow tubes of the tubing unit are combined to one outflow tube, via the at least one tubing connector, before entering into the casing of the tubing interface unit (e.g. at the patient end of the tubing interface unit).

In particular, the carrier unit, for example, comprises a receiving aperture (an opening) to cradle (or to hold) the heater unit when not in used.

In particular, the receiving aperture formed at the outer periphery of the housing at a top position thereof.

In particular, the flushing system further comprises a power source provided by a battery unit or a power plug.

In particular, the flushing system is configured to be powered by the battery unit when the system is being transported.

In particular, the control unit may provide at least one of the control signals on the basis of the obtained filling level of the buffer volume and/or a buffer container level, to thereby control the second pumping unit and adjust the effusion liquid rate (e.g., pumped volume) from the buffer container. Hence, the buffer container may collect effusion liquid until the outflow pumping device is allowed to pump. Accordingly, a steady flow of effusion liquid may be generated in a controlled manner. As a consequence, subsequent analysis means may perform precise measurements in/at the effusion liquid. The control unit may be configured to control the second pumping unit in order to pump a predetermined, i.e., a known, volume on the basis of said control signals. For example, the control unit may be configured to control the outflow pumping devices of the second pumping unit in order to pump a volume of, e.g., 30 ml, which may be referred to as a batch. However, any other volume may be pumped on the basis of the control signals of the control unit. Accordingly, the second pumping unit may be operated in a discontinuous manner by the control unit. Additionally or alternatively, the first pump device may be controlled by a control signal being based on an amount of outflow liquid.

In particular, the second pumping unit (i.e. the outflow pumping devices) may comprise a peristaltic pump. It may additionally or alternatively be conceivable, that the first pump device may comprise a volumetric pump, a membrane pump, an impeller pump and/or a syringe pump device including a syringe pump and, optionally, at least two valves. In particular, the outflow pumping devices may be any suitable pump device for effusion liquid (with or without valves, e.g., separate valves), and is preferably of a type that allows pumping at a relative accurate flow rate (e.g., that allows accurately pumping of (certain) volumes). The peristaltic pump or the syringe pump, for example, may be configured for gently conveying of sensitive material such as blood cells, which could be destroyed by rapidly moving or rotating elements, which might be part of other kinds of pumps. The peristaltic pump may perform a continuous and/or a discontinuous pumping action. The syringe pump may perform a discontinuous pumping action, wherein the pumping action of the syringe pump has a suction step and a discharging step. In the suction step, a first valve disposed upstream from the syringe pump is opened and a second valve disposed downstream from the syringe pump is closed, and, in the discharging step, the first valve is closed and the second valve is opened.

In particular, a method for irrigating a patient body, especially the pleural cavity and/or the pericardial cavity, using the flushing system as described under the above comprises the steps of:
inserting a tubing assembly into the pleural cavity and/or pericardial cavity,
optionally, priming the flushing system,
regulating flow parameters of infusion fluids via the user interface,
monitoring pleural cavity conditions and/or pericardial conditions using the integrated sensors, and
optionally adjusting an infusion flow rate based on real-time feedback.

Following an open-heart surgery, there may be some residual air in the space around the heart when closing the patient, but this poses no danger as it is not within the circulatory system (cardiopulmonary bypass circuit). Additionally, after surgery, the pericardial cavity is connected to drainage systems that have a negative pressure of −15 to −20 cmH2O to thereby ensure the removal of any air from the pericardial cavity via the drain tube(s).

In particular, air may be present in the inflow tubes when the flushing system starts. However, the infusion fluid (e.g., saline fluid) pushes this air to the patient's pericardial cavity, where it is then removed by the vacuum system integrated into the system.

The priming step, for example, is conducted by priming the inflow lines by introducing the infusion fluids at high-flow rates into the system, e.g. a flow of 1000 ml/h for a specific time, e.g. 1 or 2 min.

The flow rates and the duration of the priming step are determined depending on the length and diameter of the inflow tubes. For example, in cases where the inflow tubes are about 2.5 m, the priming duration may be about 8.5 minutes when inflow rates are low e.g. 100 ml/hr. This is not ideal for a flushing system which is connected to the patent.

The adoption of elevated priming flow rates is considered safe because the priming flow initiates only when an active and correct vacuum functioning is in place. It has been determined that the introduction of approximately 18 ml of air during the priming process does not raise safety concerns, given the existing air presence in the pericardial cavity.

The priming process guarantees an air-free infusion of fluids into the patient, preventing the introduction of air into the pericardial cavity and/or pleural cavity. In this way it is ensured that the flushing system is correctly set up and free from air or contaminants, which is vital in a wide range of medical procedures and treatments.

It is further an object of the present invention to provide for components for a flushing system, such as a measurement unit for analyzing the effusion fluids removed from a body cavity and a multi-lumen tubing set configured to be placed into a body cavity at one end and to be connected to a flushing system at another opposite end. These further aspects of the present invention will be described in the following.

According to another aspect of the present invention, a measurement unit is provided that is configured to be used (linked) with a flushing system. For example, the flushing system is configured to flush at least one body cavity with an infusion fluid.

In particular, the measurement unit is configured to be operably connected to the flushing system. e.g. a carrier unit (e.g. a console) of the flushing system.

In particular, the measurement unit comprises (e.g. is embodied as) a disposable unit or a single use unit, e.g. a cartridge unit.

In particular, the measurement unit comprises fluid, electrical and/or mechanical connections configured to be coupled to the corresponding connections included in the carrier unit of the flushing system.

In particular, the cartridge unit is configured to be fluidly connected to the flushing system for delivering the infusion fluids to the patient and/or for analyzing the removed effusion fluids from the patient.

The flushing system, for example, comprises a console, including an inflow unit for accommodating infusion fluid and a collection unit for collecting effusion fluids, and a tubing unit in fluid communication with a patient.

In particular, the measurement unit is configured for separately analyzing the effusion fluids removed, via the flushing system, from the body cavities e.g. the pleural cavity and the pericardial.

In particular, the measurement unit comprises at least two intermediate fluid containers (e.g. two buffer containers) configured to retain the effusion fluids removed from the body cavities, e.g. in an intermediate position before discharging the same into a collection unit of the flushing system.

In particular, each of the intermediate fluid containers are configured to establish fluid connection with a specific body cavity, enabling distinct analysis of the effusion fluids remover therefrom.

More particularly, the measurement unit comprises a first intermediate fluid container configured to receive the effusion fluids from a first body cavity, e.g. the pleural cavity, and a second intermediate fluid container for receiving the effusion fluids from a second body cavity, e.g. the pericardial cavity.

In particular, the first and second intermediate fluid containers comprise an inlet in fluid communication with one outflow tubes of the tubing unit (for receiving the effusion fluids from the patient), and a first outlet in fluid communication with outflow pumping devices of the second pumping unit.

In particular, the measurement unit further comprises a first pumping unit (e.g. an inflow pumping unit) for pumping the infusion fluids into the body cavities.

In particular, the carrier unit comprises the second pumping unit (e.g. an outflow pumping unit or a suction pumping unit) for removing the effusion fluids from the body cavities (e.g. the pericardial cavity and/or pleural cavity).

In particular, the measurement unit comprises an analysis chamber (e.g. a cuvette) configured to collect the effusion fluid for analysis via a sensor unit.

In particular, the second pumping unit is in fluid communication with the analysis chamber and configured to alternately coordinate (i.e. to separately direct), for example via a connecter element, the flow of the effusion fluids from the first intermediate fluid container and the second intermediate fluid container into the analysis chamber for further analysis.

Alternatively, the flushing system may comprise two sensors, each fluidly connected to the second pumping unit and configured to operate independently for analyzing the effusion fluids removed from the corresponding body cavities.

In particular, the components of the measurement unit (e.g. the intermediate containers, the first pumping unit, tubes) are configured to be fixed onto a support element (or a mount plate) by appropriate mechanical means or adhesives or glues.

In particular, the support element defines a mounting surface spanning from an upper edge to a lower edge.

For example, the second pumping unit comprises two outflow pumping devices that are configured to remove the effusion fluids, respectively, received from the pericardial cavity and the pleural cavity of the patient.

For example, the first pumping unit comprises three inflow pumping devices commonly disposed on an axis perpendicular to the mount plate.

In particular, the measurement unit is designed to be incorporated into a flushing system so that the flow of effusion fluids within the measurement unit, at least in part, is in the direction of gravity.

According to yet another aspect of the present invention, a smart tubing assembly for a flushing system is provided.

The flushing system, for example, is configured to flush at least one body cavity (e.g. the pericardia cavity and/or pleural cavity). The flushing system, for example, comprises a console, including an inflow unit for accommodating infusion fluid and a collection unit for collecting effusion fluids, and a tubing unit in fluid communication with a patient.

In particular, the tubing assembly comprises a plurality of tubes that is configured to be placed into a body cavity. For example, the tubing assembly comprises a set of tubing (e.g. a bundle of tubes) each comprising various single-lumen tubes.

Alternatively, the tubing assembly comprises a multi-lumen tube that that is configured to be placed into a body cavity. For example, the multi-lumen tube comprises a single tube including multiple lumina for inflow, pressure measurement and outflow

In particular, the tubing assembly is held in a specific, packed arrangement.

In particular, the tubing assembly is configured to be coupled to (to be used with) a flushing system, e.g. a tubing unit of the flushing system, which is configured to carry the effusion fluids.

In particular, the tubing assembly is configured to establish fluid communication with a patient, in particular a body cavity.

In particular, the tubing assembly comprises a proximal end configured to be connected to the patient, and a distal end configured to be connected to a flushing system.

In particular, the tubing assembly comprises a plurality of inflow tubes, each configured to carry infusion fluids or air to a patient, and at least one outflow tube configured to carry (drain) fluids from the patient.

In particular, the tubing assembly comprises a sleeve element (an outer tubing or a protective cover) that is configured to enclose in part the tubing assembly (including the plurality of inflow tubes and the at least one outflow tube) along a predetermined length of the tubing assembly.

The sleeve element is configured to hold the plurality of inflow tubes and at least one outflow flow tube together in a sealing manner, facilitating a leak-free passage and placement of the tubing assembly through the incision.

For example, the sleeve functions as a safety measure to seal the gaps (openings) between the tubes in the tubing assembly, via which fluid can leak out and air can leak in the patient, endangering the safety of the patient.

In particular, the sleeve element comprises an outer periphery wall defining an inner volume or space that is configured to enclose the multi-lumen tubing set (including the plurality of inflow tubes and the at least one outflow tube).

In particular, when the multi-lumen tubing is enclosed by the sleeve element, center points of the at least one outflow tube and the two of the inflow tubes are positioned along a common axis within the sleeve element.

In particular, two of the plurality of inflow tubes are connected together, forming a twin inflow tube.

For example, one of the inflow tubes in the twin-inflow tube can be an air tube connected to an infusion fluid tube. Such twin arrangement of the inflow tubes prevents the air tube from blockage via blood clots or tissues.

This provides advantageous support to the air channel through the inflow tube, ensuring its cleanliness. In other words, the flow of the infusion fluid prevents unwanted clogging of the air tube by small clots or tissues.

In particular, the at least one outflow tube is disposed between the plurality of inflow tubes.

For example, in the tubing assembly, e.g. at a position where the tubes are held by the sleeve, the at least one outflow tube is disposed in a central position, sandwiched between, at least, two of the inflow tubes.

In particular, the at least one outflow tube is disposed between the twin inflow tube at one side and one other inflow tube at other opposite side.

In particular, one of the twin inflow tubes is configured to carry air (i.e. forming an air tube). In this way, the air channel is supported by the second inflow channel, thereby preventing its blockage.

In particular, the at least one outflow tube has dimeter larger than the plurality of inflow tubes.

In particular, the plurality of inflow tubes is of identical or different diameters.

For example, the at least one outflow tube has an outer diameter between 8 to 10 mm, preferably 9 mm and defines a lumen having a diameter of 5 to 7 mm, preferably 6 mm.

The plurality of inflow tubes, for example, each defines a lumen with a diameter of 1 to 3 mm, preferably 1.5 mm. Each of the inflow tubes has an outer diameter between 2 to 4 mm, preferably 3 mm.

In particular, the tubing assembly further comprises a filler material configured to be disposed within the sleeve element (i.e. in the hollow chamber) to thereby fill-in spaces available within the hollow chamber, which are not occupied by the arrangement of the plurality of inflow tubes and the at least one outflow tube.

In this way, the gaps between the plurality of inflow tubes and the at least one outflow tube contained in the sleeve element (i.e. arranged in the sleeve element) can be easily filled with the filler material. The filling process is achieved without being constrained by the shape or size of the tubes and the sleeve element.

Advantageously, by way of the sleeve element and the filler material, it is possible to provide support for the plurality of inflow tubes and the at least one outflow tube, securely anchoring them in a sealing manner. This, in turn, results in preventing undesired leakage, such as air or fluids, from the patient into the surrounding environment.

For example, a sleeve element or an outer tubing is configured to be fitted over the plurality of inflow tubes and the at least one outflow tube, over a certain length, at a proximal end, i.e. at a location proximate to the patient, in particular proximate to an incision location (outside the patient's body).

Alternatively, the sleeve element, for example, is produced by injection moulding and comprises at least two portions that are configured to be attached together, e.g. assembled together. The at least two portions are designed and configured to hold (accommodate) and secure the tubes of the tubing assembly in place.

In this way, advantageously, the tubes exiting the patient body are tightly hold together in a sealing manner.

For example, the tubing assembly comprises three inflow tubes carrying infusion liquids into the patient (e.g. a body cavity), one pressure tube (air tube) carrying air, and one out flow tube carrying effusion fluids from the patient.

In particular, the tubing assembly further comprises a cap disposed at a distal end of the tubing assembly (i.e. far from the patient, near to a flushing system). Preferably, the cap allows to pull a tubing assembly through an incision out of a body cavity. In other words, the cap helps to guide the tubing assembly, i.e. placing from the (still open) body cavity, through the incision, out of the body (from inside-out).

### BRIEF DESCRIPTION OF THE FIGURES

It is shown in

FIG. 1: a schematic overview of a flushing system;

FIG. 2: a schematic overview of a carrier unit included in the flushing system;

FIG. 3 a perspective view of the carrier unit without the inflow, measurement and collection units;

FIG. 4 a perspective view of a tubing interface unit for the flushing system;

FIG. 5 top and bottom perspective views of the tubing interface unit;

FIG. 6 a perspective view of two alternative designs for a collection unit for the flushing system;

FIG. 7 a schematic front view of a measurement unit for the flushing system;

FIG. 8 a schematic rear (reverse) view of a measurement unit for the flushing system;

FIG. 9 a perspective view of a measurement unit for the flushing system;

FIG. 10 a schematic view of a tubing assembly for the flushing system; and

FIG. 11 a schematic view of the flushing system.

### DETAILED DESCRIPTION

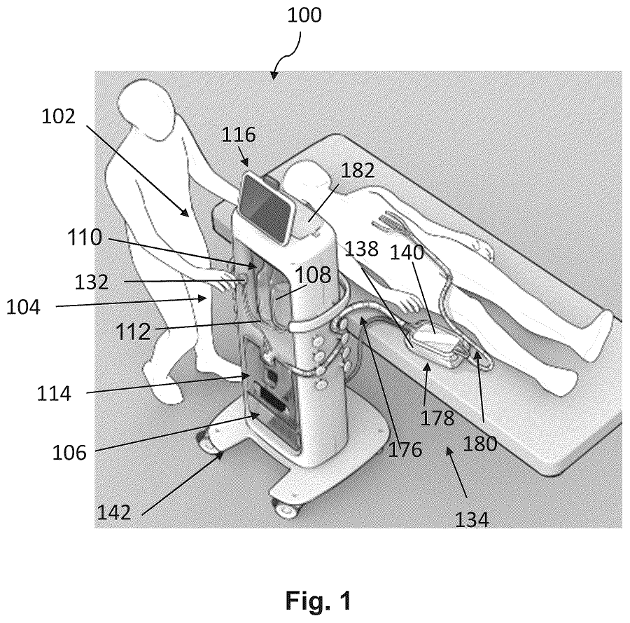

FIG. 1 shows a schematic overview of a flushing system 100 being in fluid communication with a patient. The flushing system 100 is configured for flushing a body cavity, e.g. the pericardial cavity or the pleural cavity.

The flushing system 100 comprises a carrier unit 102 which is configured to be moved, e.g. configured to be transported and brought close to the patient's bed. For example, the carrier unit 102 in the form of a cart or console unit.

The flushing system 100 further comprises an inflow unit 104 and a collection unit 106.

The inflow unit is configured to provide a flushing solution (i.e. an infusion fluid) for the patient.

In particular, the inflow unit 104 comprises at least one fluid reservoir 108, e.g. in the form of a fluid container or vessel for holding the flushing solution typically sterile saline or another appropriate fluid.

The flushing system 100 further comprises a measurement unit 114 that is operably connected to the inflow unit 104 and the collection unit 106.

In particular, the measurement unit 114 is in fluid and/or electrical communication with the inflow and collection units.

The measurement unit 114 is configured to deliver the infusion fluid to the patient, to receive the effusion fluids from the patient, and to analyse the effusion fluid.

The inflow unit 104 further comprises at least one inflow tube 112 that is configured to carry (or convey) the infusion fluid via the measurement unit 114 to the patient. In other words, the inflow unit is in fluid communication with the measurement unit via the at least one inflow tube.

For example, the carrier unit 102 comprises electronics, at least one measurement unit and/or at least one processing unit. In other words, the carrier unit 102 comprises re-usable components, such as a level sensor, peristaltic pumps for pumping outflow from buffer volumes towards a hematocrit sensor. The measurement unit 114, for example, comprises disposable components, such as a cuvette for the haematocrit sensor.

In particular, the measurement unit is disposed in an intermediate position between the inflow unit and the collection unit. For example, the measurement unit is arranged downstream from the inflow unit along a longitudinal axis (i.e. in the direction of gravity) of the flushing system.

The inflow unit 104, the measurement unit 114 and the collection unit 106 all are configured to be disposed in the carrier unit 102.

In particular, the carrier unit 102 comprises a base unit 142 configured to facilitate movements of the carrier. For example, the base unit comprises driving elements, such as wheels for moving the carrier unit 102.

The flushing system 100 further comprises at least one first weighing unit 110 that is included in the inflow unit 104. The at least one first weighing element is configured to measure a weight of the flushing or infusion fluids which are to be delivered to the patient.

The at least one first weighing unit 110 is also configured to retain the at least one infusion fluid container 108.

In particular, the at least one first weighing unit 110 is configured to be coupled to the carrier unit 102 in a floating arrangement via a shock mounting.

In this way, the at least one first weighing unit is isolated (i.e. mechanically decoupled) from external vibrations and shocks applied, for example, to the carrier unit. The floating arrangement of the shock mounting allows the at least one first weighing unit to be moved freely within the carrier unit 102, thereby reducing the impact of external vibrations and shocks on the first weighing unit.

For example, the at least one first weighing unit 110 comprises a case (not visible in FIG. 1) for retaining (or enclosing) the at least one infusion fluid container 108.

The case (casing or a support structure) is, for example, of a shape and design suitable to hold the at least one fluid container. For example, the casing includes an inverted U-form or a hollow cup for retaining the at least one fluid container 108 therein.

In particular, the flushing system further comprises a second weighing unit (not shown in FIG. 1) which is included in the carrier unit in close proximity with the collection unit 106 for measuring the infusion fluids contained in the collection unit 106.

The flushing system 100 further comprises a tubing unit 134, which tubing unit provides for a fluid communication between the carrier unit 102 and the patient.

The tubing unit 134 is configured to be connected (directly) to the measurement unit 114 or to be connected, via the measurement unit, to the collection unit 106.

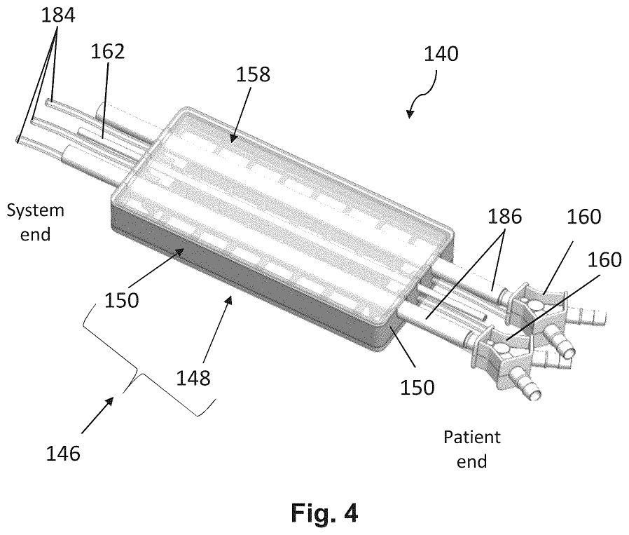

The tubing unit 134 comprises a plurality of inflow tubes 184 for carrying the infusion fluids from the flushing system 100 to the patient, and a plurality of outflow tubes 186 for carrying the effusion fluids from the patient to the system (see FIG. 4).

In particular, the tubing unit 134 includes a first tubing section 176 (e.g. with an end) that is connected to the flushing system 100, i.e. the measurement unit, and a third tubing section 180 (e.g. with an opposite end) that is connected to the patient.

The tubing unit 134 further comprises a second tubing section 178 that is disposed between the first and second tubing sections.

The tubing unit 134 may further comprise a tubing assembly 220, e.g. a bundle of inflow tubes and outflow tubes joined together, disposed in the third section 180 thereof, which is configured to be fluidly coupled to the patient.

The flushing system 100 further comprises a heater unit (or a heating unit) 138 for heating, at least partly, the tubing unit 134, e.g. the second section thereof 178. For example, the heater unit 138 is configured to heat the infusion fluids carried by the plurality of inflow tubes of the tubing unit 134.

The heater unit 138 comprises a plurality of heating elements. The heater unit 138 or the heating elements further comprises a contact surface that is configured to get heated by the heating elements. The contact surface is configured to provide a heated surface for heating the infusion fluids carried by the inflow fluids.

In particular, the flushing system 100 comprises a temperature control unit operably connected to the heater unit 138. The temperature control unit, e.g. the heater unit 138, comprises a sensor for measuring the temperature of the heating elements of the heater unit 138 and a temperature control unit for regulating the temperature.

For example, the temperature control unit is configured to determine the temperature of the fluid being in contact with the contact surface based on factors such as time, the surface dimensions and/or the flow rate of the fluid along the surface.

Alternatively, the temperature control unit comprises a temperature sensor to measure a temperature of infusion liquid and a control unit to control the heating unit for heating the infusion fluids to the desired temperature, e.g. body temperatures (in a range of 36° C. and 38° C., preferably 37° C.).

The desired temperature for the infusion liquid may also be adjusted to the actual body temperature of the patient or may be adjusted in such a manner that it heats or cools down the heart and its surroundings when clinically appropriate. In particular, the heating may be necessary if a patient is cooled down after an operation, which is the case in the majority of patients, and otherwise, the cooling of, e.g., the heart, which may be necessary in case of certain heart rhythm disturbances or when the patient is in a “cooling protocol” which is needed after serious adverse events peri-operatively like a period of total circulatory collapse and/or cardiopulmonary resuscitation.

The carrier unit 102, for example, further comprises a receiving element 182 (including a receiving opening or grooved surface) to hold or (to cradle) the heater unit 138 when not in used (see FIG. 1).

In particular, the flushing system 100 further comprises a tubing interface unit 140 that is configured to retain (i.e. to hold in place), at least partly, the tubing unit 134 which is in fluid communication with the patient (and which is configured to carry the infusion fluids and the effusion fluids).

For example, the second section of the tubing unit 134 is configured to be held by the tubing interface unit 140.

In particular, the tubing interface unit 140 is configured to be connected to the heater unit, wherein a plurality of inflow tubes that is included in the tubing unit 134 is configured to be heated via the heater unit 138.

Also included in the flushing system 100 is a user interaction unit 116 (a user interface unit), allowing a user to monitor flushing processes and/or physiological conditions of the patient.

For example, the user interaction unit 116 includes a user-friendly interface for healthcare providers to input and adjust parameters such as flow rate and duration of a flushing process. This can be a touchscreen or physical buttons.

In particular, the flushing system 100 further comprises a power source provided by a battery unit or a power plug.

The flushing system 100 is configured to be powered by the battery unit during transportation.

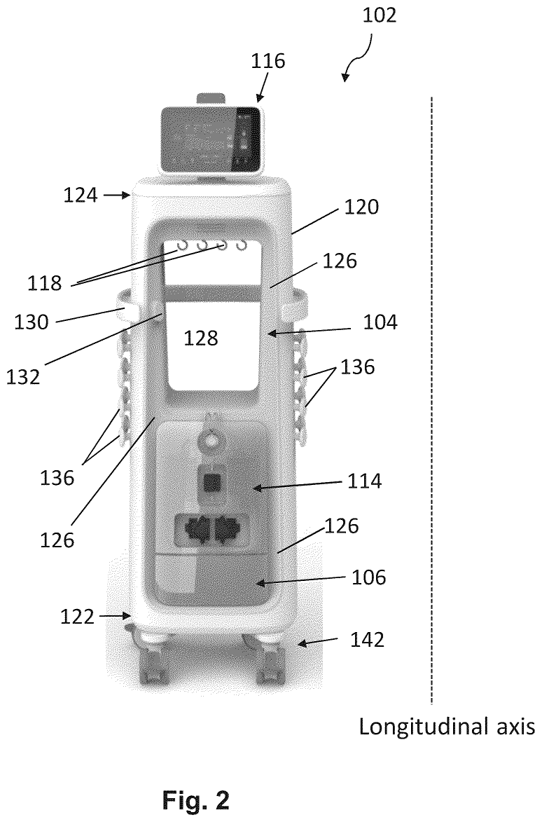

FIG. 2 shows a schematic overview of the carrier unit 102 of the flushing system 100.

In particular, at least one mounting element 118 for holding the case of the first weighing unit 110 or the at least one fluid container 108 is shown in the inflow unit 104.

The at least one mounting element 118, for example, comprises at least one mechanical fastener or snapping element configured to hold (or catch) the at least first weighting element or the at least one fluid container.

For example, the at least one mounting element is in the form of a hook, wherein the fluid container or the first weighing element is configured to be mount thereon.

In particular, the carrier unit 102 comprises a housing 120 extending along the longitudinal axis (shown in FIG. 2 by a dotted line) of the flushing system from a top position (top portion) 124 to a bottom position (a base portion) 122.

The housing 120 is configured to accommodate the collection unit 106, the measurement unit 114 and the inflow unit 104, respectively, from the bottom position 122 to the top position 124.

For example, the collection unit 106, the measurement unit 114 and the inflow unit 104 all are configured to be stacked in the carrier unit 102 (i.e. in the housing 120) in a modular arrangement along the longitudinal axis.

In particular, the housing 120 comprises a peripheral (outer) wall that extends, along the longitudinal axis, i.e. between the base and top portions.

In FIG. 2 it is shown that the driving elements, e.g. the wheels are configured to be connected to the housing of the carrier unit 102 at the bottom position 122 thereof.

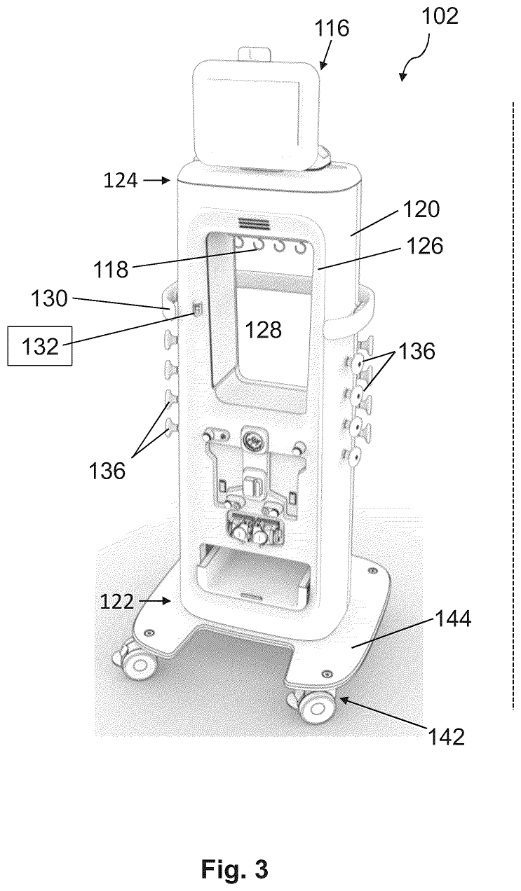

Alternatively, the base unit 142 comprises a footplate 144 (see FIG. 1 or 3) and the wheels are attached to the housing 120 via the footplate.

The footplate is dimensioned such that when it is coupled to the housing of the carrier unit 102, an outer edge (a step-like surface) is formed around the periphery of the housing.

In particular, the carrier unit 102 comprises an inner frame 126.

The inner frame 126 is arranged (or housed) in the housing 120 of the carrier unit 102 and is configured to (operably) accommodate the inflow unit, the measurement unit and the collection unit therein.

In particular, the inner frame 126 defines a hollow chamber 128 for accommodating the inflow unit 104 therein. For example, the hollow chamber 128 is formed as a pass-through chamber, e.g. with two open side-walls, wherein the inflow unit is configured to be arranged in the hollow chamber.

The at least one mounting element 118 (e.g. a hook(s)) of the inflow unit 104, for example, is (are) configured to be connected to the inner frame 126, e.g. to an inner wall of the hollow chamber such that the fluid containers are configured to be hanged thereon (i.e. in the direction of gravity as shown in FIG. 2).

As illustrated in FIGS. 1 and 2, the user interface unit 116 is configured to be attached onto the housing, preferably on an outer periphery of the housing at the top portion.

Referring to FIGS. 1 and 2, a gripping element 130 is further included in the flushing system 100.

In particular, the gripping element 130 (partly visible through the hollow chamber 128) is configured to protect the first weighting unit 110 and/or the at least one infusion fluid container 108.

The gripping element 130 is further configured to provide a grip, such as a handlebar or handgrip for the carrier unit. In this way, it is possible e.g. to hold, move, push, pull the carrier in an easy manner.

For example, the gripping element 130 comprises an arm element (e.g. a rod or bar structure) that is connected to the housing (e.g. outer peripheral wall of the housing).

The arm element extends around partly a periphery of the housing, to thereby provide a barrier (a protection or guard) at one side of the hollow chamber. In other word, the barrier is arranged in front of the one of the open side-walls.

In particular, the at least one first weighing unit is configured to be moved (e.g. via a floating arrangement within the hollow chamber) independent of the carrier unit and/or its movements.

In other words, the at least one first weighing unit 110 is configured to be connected to the inner frame 126 by way of a shock mounting mechanism to thereby mechanically decouple the at least one weighting unit 110 from the inner frame and/or housing or the carrier unit 102).

FIG. 3 shows a perspective view of the carrier unit 102 for the flushing system 100 without the measurement unit 114 and the collection unit 106 installed therein.

As shown in this Figure, the inner frame 126 comprises a through chamber near to the top portion 124 of the housing 120 for housing the inflow unit 114, a first cavity, provided in an intermediate position between the top and base positions, for receiving the measurement unit 114, and a second cavity near to the base portion 122 of the housing 120 for receiving the collection unit 106.

In particular, the measurement unit 114 and the collection unit 102 are configured to be removably fitted in the first and second cavities and in case of a failure, they can independently be replaced or repaired as necessary.

The carrier unit 102, e.g. the inner frame 126 and/or the housing 120, comprises fluid connections and/or electrical connections for the operation of the inflow unit, the measurement unit and the collection unit as may be required.

In particular, the flushing system 100 further comprises a locking element 132 for holding the at least one inflow tube of the inflow unit 104 in place (see FIGS. 1-3).

The locking element 132, for example, is connected to the carrier unit 102.

For example, the locking element 132 is connected to or formed as an integral part of in housing 120 or the inner frame 126 of the carrier unit 102.

Advantageously the locking element 132 is configured to fix a position of the at least one inflow tube 112 to prevent (i.e. block) it from moving (or displacement), e.g. when the carrier unit is being moved. The locking element 132 further serves as strain relief to avoid influencing a load cell of inflow part (unit) by weight of the tubes and other disposable components.

In FIG. 3 guiding elements 136 are also shown. The guiding elements 136 comprise a plurality of tabs or retaining elements that are disposed on the sidewalls (peripheral wall) of the housing.

The guiding elements 136 are configured to hold, at least in part, a position of the first section of the tubing unit 134 on the sidewalls.

For example, the guiding elements 136 are arranged such that the position of the tubing unit towards the carrier unit can be controlled such that the flow the effusion fluids towards the carrier unit is supported in the direction of gravity (i.e. a downward flow).

In particular, the plurality of retaining elements is arranged in a row-column configuration on the sidewalls of the housing. The retaining elements serve the purpose of guiding the tubing unit so that the flow of the fluid towards the carrier unit is prompted. This ensures that the effusion fluid moves in the direction of gravity, as the tubing unit progresses from a higher to a lower position along the longitudinal axis.

In particular, the tubing unit 134 further comprises a pressure measurement tube 162 for carrying air from the carrier unit 102 to the patient, e.g. an air tube (see FIG. 4). The pressure measurement tube extends along the first, second and third tubing sections of the tubing unit 134.

In particular, the receiving element is formed at the outer periphery of the housing of the carrier unit at the top portion thereof.

FIG. 4 shows a perspective view of a tubing interface unit 140 for a flushing system 100.

The tubing interface unit 140 is configured to partially house (accommodate) the tubing unit 134 of the flushing system 100.

The tubing interface unit 140 comprises a casing 146 including a base plate 148 and a peripheral or exterior wall 150 extending upwardly from the base plate 148. For example, the peripheral wall and the base plate are configured to be removably connected, defining a chamber to accommodate, e.g., a portion of the tubing unit.

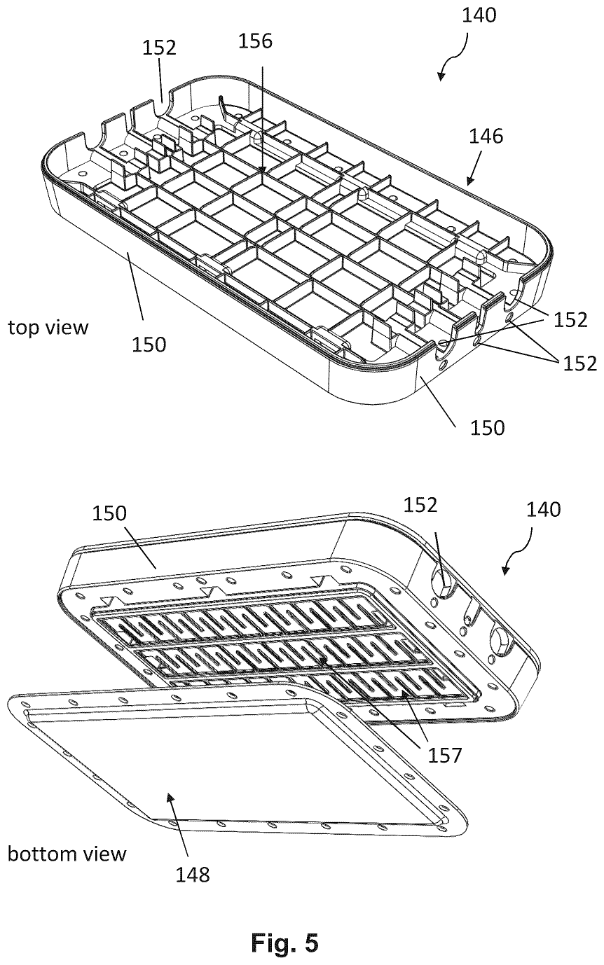

In particular, a plurality of openings 152 (see FIG. 5) is formed on the wall 150 on one side and other opposite side of the casing 146 for retaining the tubing unit 134 therein.

For example, the plurality of openings 152 is dimensioned and arranged such that the plurality of the inflow tubes and/or the plurality of outflow tubes are configured to pass therethrough from the one side of the casing to the other opposite side of the casing.

In particular, the base plate/structure 148 is configured to be connected to and to get heated via the heater unit 138.

In particular, the tubing interface unit 140 further comprises an inner structure 156 that is disposed within the casing. The inner structure 156 is configured to define a network of passages 157 (paths) for the plurality of inflow tubes to pass therethrough (visible in FIG. 5). The elongated path traversed by each inflow tube within the casing results in prolonged contact with the heater unit 138, achieving efficient heating of the infusion fluid to the desired temperature, such as body temperature.

For example, the base plate comprises a conductive (e.g. metallic material) for better transfer of heat.

The tubing interface unit 140 further comprises a lid 158 that is configured to be placed onto the casing 146 (on a free end of the peripheral wall 150) for closing the same.

For example, the tubing interface unit 140 is configured to hold the second section 178 of the tubing unit 134.

In particular, the tubing interface unit 140 is in the form of a hub that is configured to partially hold the tubing unit and to heat the plurality of inflow tubes carrying the infusion fluid.

In particular, the tubing interface unit 140 comprises a first end, for example, for receiving the plurality of inflow tubes from the flushing system 100 (e.g. a system end/side), and a second end, for example, for receiving the plurality of outflow tubes from the patient (e.g. patient end/system side).

FIG. 4 further illustrates that two tubing connecters 160 are included in the third section 180 of the tubing unit 134. Each tubing connector 160 is configured to combine, for example, two outflow tubes connected to the patient into one outflow tube.

In particular, the tubing connectors 160, each are configured to combine an outflow tube included in e.g. a tubing assembly 220 with a further outflow tube that is not part of the tubing assembly (is not included in the tubing assembly).

For example, the tubing connectors 160 are disposed at the second end of the tubing interface unit 140 (the patient side), and each are configured to combine two outflow tubes received from a body cavity of the patient (i.e. the pericardial cavity or the pleural cavity).

In particular, the plurality of inflow tubes 184 comprises, for example, three inflow tubes carrying the infusion fluid to the patient, i.e. the inflow tubes extend along the first, second and third sections 176, 178, 180 of the tubing unit 134.

The plurality of outflow tubes 186 comprises, for example, four outflow tubes, at the third tubing section 180 of the tubing unit 134, that are configured to discharge the effusion fluids from the patient.

These four outflow tubes are configured to be combined, for example, to two outflow tubes at the second tubing section, which two outflow tubes further extends through the first tubing section to the flushing system.

In particular, the plurality of outflow tubes 186 is configured to be connected together using tubing connecters 160.

FIG. 5 shows top and bottom perspective views of the tubing interface unit 140.

In this Figure, the inner structure 156 that is arranged within the casing 146 of the tubing interface unit 140 is indicated.

The inner structure 156 includes partitioning elements configured to define a network of passages 157, e.g. a separate passage or channel for each of the plurality of inflow tubes passing therethrough (visible in the bottom view).

For example, the inner structure 156 of the tubing interface unit 140 is in the form of a labyrinth including a plurality of channels extending from the system end to the patient end.

Each of the partitioning elements is configured to define a separate passage (or channel) for each of the plurality of inflow tubes, which passage is configured to define a meandering path with several turns between the system end and the patient end of the tubing interface unit 140.

In this way, the length of the path leading to the other end of the tubing interface unit 140 is significantly increased. This, in turn, enhances the contact surface between the channels (i.e. the bottom side of the channels) and the heating unit 138, allowing the infusion fluid carried by the inflow tubes to get heated to a desired temperature.

In particular, the plurality of openings 152 comprising a first row of openings for receiving the plurality of inflow tubes are formed on the peripheral wall 150 at a position near to the base structure 148 of the casing 146.

The plurality of openings 152 further comprises a second row of openings for receiving the plurality of outflow tubes. The second row of openings are arranged on the peripheral wall 150 at a position near to the free end thereof.

In other words, the second row of openings are disposed on the peripheral wall 150 upstream from the first row of openings.

The dimensions of the openings are adapted to the dimensions of the inflow and outflow tubes. The second row of openings are, for example, larger than the first row of openings.

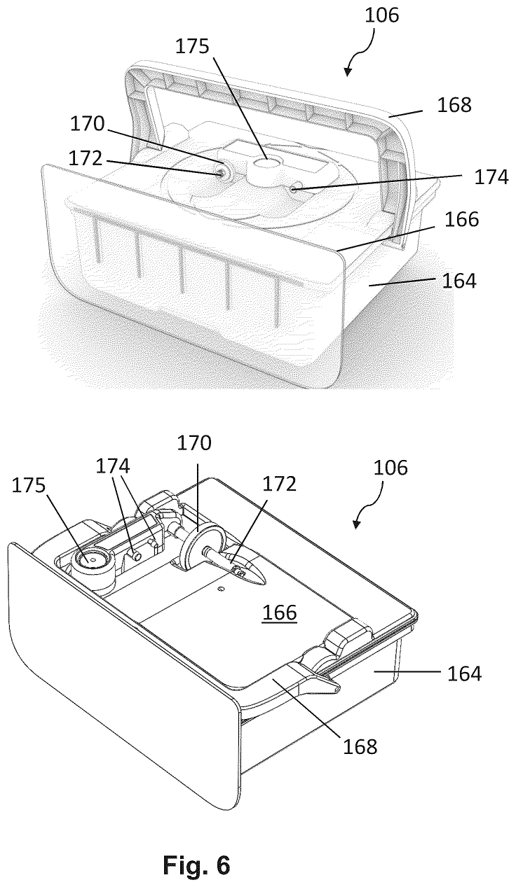

FIG. 6 shows a perspective view of two alternative designs of a collection unit 106 for a flushing system 100. Similar components are shown with identical reference signs.

In both designs, the collection unit 106 is configured to be removably disposed within the carrier unit 102 of the flushing system 106. For example, the collection unit 106 is configured to be slide into in the inner frame of the carrier unit 102.

In particular, the carrier unit 102is configured to measure, via the second weighing unit 111, the weight of the effusion fluids collected from the patient.

The collection unit 106 comprises a waste container 164 (e.g. a reservoir) that is configured for containing the effusion fluids collected from the patient.

Further, a cover element 166 is provided that is configured to be connected (attached) to the waste container 164 in a sealing manner.

For example, the collection unit 106 may further comprise a handle 168 that is hinged to/pivotally connected to the waste container 164 and/or the cover element 166.

The handle 168 is used for carrying and transportation of the collection unit 106.

In particular, at least one filter element 170 is included in the collection unit 106.

The at least one filter element 170 is configured for capturing biological components, thereby preventing biological contaminations (biological pollutants) to enter into atmosphere via the vacuum pump of the carrier unit 102.

The collection unit 106 further comprises a first coupling element 172 that is configured for connection to a vacuum pump disposed in the carrier unit 102, i.e. a suction line.

For example, the at least one filter element 170 is configured to be disposed downstream from the first coupling element 172 (in the direction of the flow of the effusion fluids).

The collection unit 106 is further comprises at least one second coupling element 174 that is configured for connection to the measurement unit 114, e.g. to a discharge tubing of the measurement unit.

In particular, the waste container 164 has a volume of about 4 or 5 L.

In particular, the collection unit further comprises an overflow protection valve 175 for protecting the coupling elements against fluid overfill.

In particular, the filter element 170, the first and second coupling elements 172, 174 and the over flow protection valve 175 are configured to be disposed in the cover element 166 of the collection unit 106.

For example, the first coupling element comprises a Luer connector, that is configured to be connected to vacuum of the carrier unit.

In the collection unit 106 shown at the bottom of FIG. 6 comprises a pair of second coupling elements 174 for connection to the measurement unit 114.

Further, the arrangement of the components of the collection unit is different. The filter element 170, the first and second coupling elements 172 and 174, and the protection valve 175 are disposed at one side of the collection unit, i.e. the cover element 166.

In the alternative design of the collection unit 106 shown at the top pf FIG. 6, the components of the collection unit 106 are centrally arranged in the cover element 166 of the collection unit.

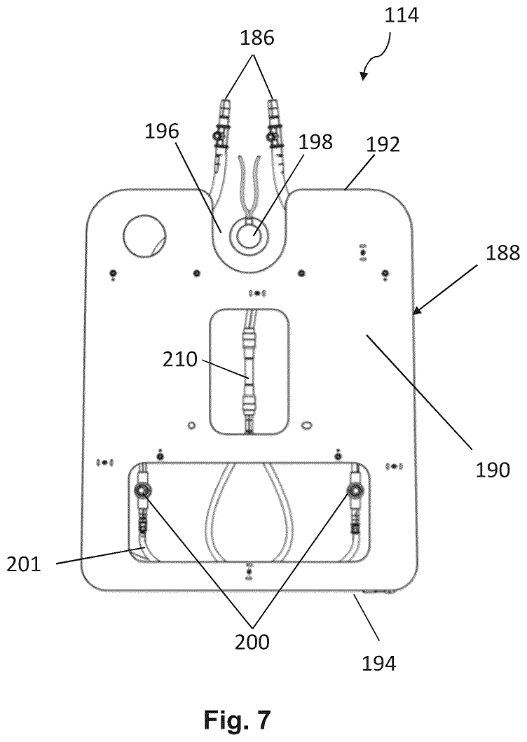

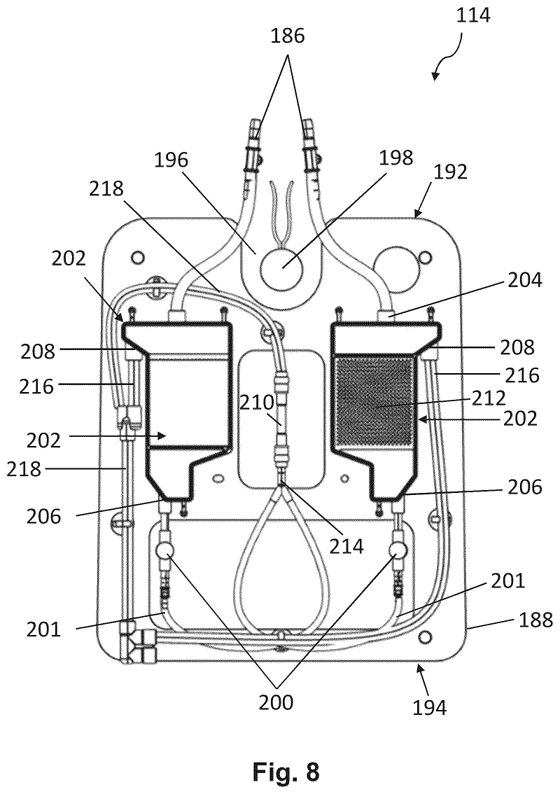

FIGS. 7 and 8 show a measurement unit 114 for the flushing system 100, respectively, from the front view and the reverse (rear) view.

For example, the measurement unit 114 is embodied in the form of a disposable unit (the so-called cartridge) that is configured to operably coupled a flushing system, e.g. to the carrier unit 102 of the flushing system 100.

For example, the measurement unit includes fastening or coupling means such as plugs, clips and socket connectors, facilitating an easy assembly and mounting of the unit to the flushing system. That is the measurement unit is designed to be effectively linked to the system and operate as intended.

In particular, the measurement unit 114 comprises components (such as pump devices, sensors, valves, fluid containers, tubing, and tubing connectors that are assembled (operably joined together), providing for one functional unit. This functional unit is configured, for example, for pumping the infusion fluids to the patient, for intermediate retaining of the effusion fluids from the patient, for analyzing the effusion fluids, and for guiding the effusion fluids to a collection unit.

In particular, the measurement unit 114 comprises a support element 188 (e.g. a mount plate or a support plate) that is configured to support a position of the components of the measurement unit. In other words, the components of the measurement unit are configured to be mounted onto the mount plate.

The mount plate comprises mechanical fasteners and coupling elements suitable to hold and to connect the components of the measurement unit (e.g. pumps, sensors and tubing) in place.

Alternatively, the support element or the mount plate comprises fluid channels and/or (fixing) elements (for holding the components of the measurement unit in place) that are/is integrated in the support element. For example, the support element and the corresponding integrated elements and channels are formed via injection molding processes.

For example, the support element 188 includes a plurality of fixing elements such as mounting clamps or clips for mounting the components of the measurement unit thereon.

In particular, the support plate 118 defines a mounting surface 190 including an upper edge 192 and a lower edge 194, wherein the measurement 114 is configured to guide the effusion fluids from the upper edge to the lower edge.

The measurement unit 114 is configured to be included (installed) in the flushing system 100, e.g. installed in the carrier unit 102, so that the flow of effusion fluids within the measurement unit 114 along the longitudinal axis (in the direction of gravity) is partly governed.

In other words, the components of the measurement unit 114 are arranged on the mounting surface 190 such that the effusion fluids enter the measurement unit 114 at the upper edge 192 and exit the same at the lower edge 194.

In particular, the measurement unit 114, i.e. the support plate 188, comprises an entrance opening 196 for accommodating a first pumping unit 198 (e.g. an inflow pump unit) for pumping the infusion fluids received from the inflow unit 104 towards the patient.

For example, the entrance opening 196 is disposed at the upper edge 192 of the support plate 188.

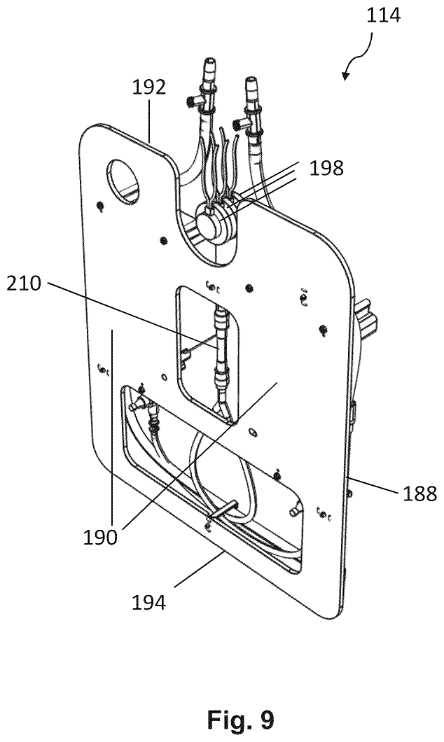

The first pumping unit 198, for example, comprises a plurality of pumping devices 198 (see FIG. 9). The plurality of pumping devices, i.e. the inflow pumps, are configured to provide (to pump), via the tubing unit 134, a plurality of infusion fluid streams for the patient.

In particular, the first pumping unit 198 is configured to be coupled to the tubing section 134 and to pump, via the inflow tubes 184 of the tubing section 134, the infusion fluids that are received from the inflow unit 104 toward the patient.

In FIG. 8 it is further shown that the measurement unit comprises sample ports 200 for taking samples of outflow fluid removed from the patient as appropriate. This feature is an optional feature.

The flushing system, e.g. the carrier unit, further comprises a second pumping unit (i.e. an outflow pumping unit) that is configured to discharge the effusion fluids from the measurement unit 114. The second pumping unit is in fluid communication with the measurement unit 114 via connector tubes 201.

For example, the second pumping unit is configured to be disposed, e.g. in the carrier unit 102. The second pumping unit comprises, for example, two outflow pumping devices (e.g. including peristaltic pumps) that are configured to remove the effusion fluids, respectively, received from the pericardial cavity and the pleural cavity of the patient.

The measurement unit 114 (e.g. the cartridge unit) further comprises at least two intermediate fluid containers 202 (e.g. two buffer containers) that are configured to store the effusion fluids. This enables storing the infusion fluids in an intermediate position in the flushing, i.e. prior to collecting the infusion fluids in the collection unit.

More particularly, each of the buffer containers 202 is configured to receive the effusion fluids from a body cavity, e.g. the pericardial cavity or the pleural cavity, and to discharge the effusion fluids when a certain amount of fluids is accumulated therein.

For example, one of the fluid containers 202 is configured to receive the effusion fluid from the pericardial cavity and the other one of the fluid containers 202 is configured to receive the effusion fluid from pleural cavity.

The intermediate fluid containers 202 are, for example, symmetrically positioned on the mounting plate 188 near to the upper edge 192 of the mounting surface 190. Other unsymmetrical configurations are also possible, e.g. one closer to the to the upper edge and the other one closer to the lower edge.

The buffer containers 202 are configured to be fixed onto the mount plate by suitable adhesives or glues or mechanical means.

The intermediate fluid containers 202, each comprises an inlet 204 and a first outlet 206 and a second outlet 208.

The inlet 204 is in fluid communication with one outflow tube of the plurality outflow tubes 186 of the tubing unit 134 (for receiving the effusion fluids from the patient), and the outlet 206 is in fluid communication with one of the outflow pumping devices of the second pumping section.

In addition, a level sensor (not shown) is included behind each of the buffer containers 202, e.g. near to the first outlet 206, which is configured for measuring a level of the effusion fluids within the buffer container 202.

In particular, the level sensors are configured to measure fluid levels in the buffer containers 202. When a level sensor measures a specific fluid level in one of the buffer containers 202, the corresponding outflow pumping device is configured to expel (or to displace) the accumulated fluid (e.g. 8 ml, 10 ml or 12 ml) through the first outlet 206 from that buffer container 202.

FIG. 8 further shows that outputs of the two outflow pumping devices are in fluid connection with a sensor unit (i.e. a hematocrit sensor) that is disposed in the carrier unit 102.

The sensor unit is configured to analyze the effusion fluids removed from the pericardial cavity and/or the pleural cavity.

In particular, the sensor unit is a hematocrit senor including a cuvette 210, disposed in the measurement unit 114, for containing the fluid effusions for further analysis.

In particular, the measurement unit 114 further comprises a connector element 214 (e.g. a Y-connector) that is configured to fluidly connect the second pumping unit with the cuvette 210.

The two outflow pumping devices of the second pumping unit are configured to operate asynchronously, i.e. not at the same time. In this way, the effusion fluids from the pericardial cavity and the pleural cavity are separately delivered through the Y-connector 214 to the cuvette for further analysis by the hematocrit sensor.

In particular, a filter 212 (e.g. a clot trap) is disposed within each buffer container 202 for trapping blood clots larger than a determined size and preventing them from exiting the buffer container 202 (in FIG. 8 only one of the filters are shown).

For example, the filters 212 are configured to be disposed in a direction along (in parallel) to the flow direction of the effusion fluids. That is, the filters 212, when in operation, are aligned in parallel to the longitudinal axis of the flushing system 100.

By way of the vertical (upstanding) arrangement of the filters 212 within the buffer containers, the performance of the filters 212 is improved and the occlusion (blockage) of the filter by the blood clots, preventing the flow of the effusion fluids through the buffer container is reduced.

In particular, each of the filters 212 is configured to filter particles or components in the effusion fluid having a diameter of ≥2.5 mm.

For example, the buffer containers 202, each define for example a volume of 200 to 300 ml.

When operating the flushing system, small blood clots might have been entered in the buffer containers through the outflow tubes. By way of regular discharging of the fluids from the buffer containers 202 and/or the provision of the filters within the buffer containers, the aggregation of the small blood clots in the further (downstream) part of the flushing system is prevented. In other words, it is ensured that no larger aggregates of the blood clots are formed.

Further, the aggregation of the blood clots in the cuvette 210 of the measurement unit 114 is mitigated as well.

In particular, the measurement unit 114 further comprises two safety elements 216 (e.g. safety overflow exits or tubes), each in fluid communication with one of the buffer containers 202.

The safety elements are overflow exits of the buffer containers 202 and configured to discharge the effusion fluids from the second outlet 208 of the buffer containers, for example, when the buffer container is filled up to a predetermined volume.

For example, the two safety exits are in fluid communication overflow tubes 216 that allow the effusion fluid to exit the buffer containers 202. This can happen, for example, in situations where the outflow pumping devices are malfunctioning or have failed, and the effusion fluids cannot be pumped out through the first outlet 206 of the buffer containers 202.

The overflow tubes are configured to fluidly be connected to the collection unit 106. That is, it is possible to evacuate the excess effusion fluids, via the outflow tubes 216, into the collection unit 106.

In particular, the measurement unit 114 further comprises a discharge tube 218 the is configured to fluidly connect the cuvette 210 of the sensor unit to the collection unit 106 of the flushing system 100. In other words, the discharge tube 218 is configured to discharge (carry) the effusion fluid from the cuvette to the collection unit 106.

For example, in operation, the effusion fluid is pumped from buffer container 202 to the cuvette 210, where it pauses during analysis by the hematocrit sensor in the carrier unit 102. After analysis, the effusion fluid is pumped towards the collection unit 106 via discharge tube 218.

In particular, the flushing system comprises an aperture (e.g. formed on the support element) that is configured to retain and guide an air tube (a pressure line) and a vacuum tube (i.e. a vacuum line form the collection unit) towards the tubing unit 134 patient

FIG. 9 shows a prospective view of the measurement unit 114.

The first pumping unit 198 in this example comprises three pumping devices that are stacked along a common axis (e.g. an axis perpendicular to the mount plate). These three pumping devices are assembled into a pump holder (not in picture) for correct placement and ease of placing and removing the pumps all together.

In particular, each pumping device is configured to be connected to the at least one inflow tube 112 of the inflow unit 104 and one of the inflow tubes 184 of the tubing unit 134.

Each of the pumping devices are further configured to pump the infusion fluids received from the infusion fluid container 108 through the tubing unit 134 to the patient.

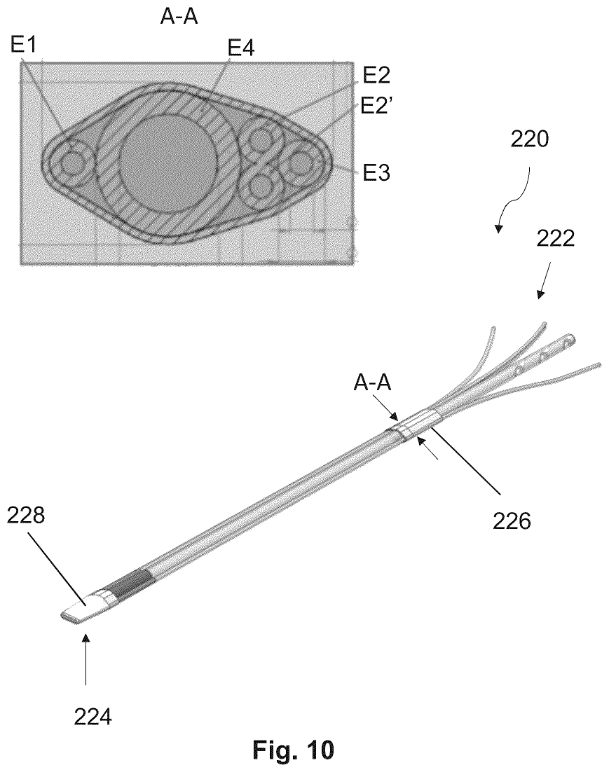

FIG. 10 shows a tubing assembly 220, especially a tubing assembly for a flushing system. The cross-sectional view labelled A-A of the tubing assembly, indicating a multi-lumen tubing set, is also depicted in the upper-left corner of FIG. 10.

In particular, the tubing assembly 220 that is configured to be connected with the flushing system 100, especially with the tubing unit 134 of the flushing system 100.

In particular, the tubing assembly 220 is configured to provide fluid communication with the patient, in particular a body cavity.

The tubing assembly 220 comprises a proximal end 222 configured to be connected to the patient (e.g. disposed within a body cavity), and a distal end 224 configured to be connected to the flushing system 100, in particular the tubing unit 134.

In particular, the tubing assembly 220 comprises a tubing set including a plurality of inflow tubes E1, E2, E2′, E3 configured to carry the infusion fluids to the patient, and at least one outflow tube E4 configured to carry (to drain) the effusion fluids from the patient.

The plurality of inflow tubes in the tubing set may further comprise an inflow tube for carrying air E2′ (e.g. an air tube or pressure line)

In particular, the tubing assembly 220 comprises a sleeve element 226 (e.g. a protective cover) that is configured to enclose the plurality of inflow tubes and the at least one outflow tube along a predetermined length of the tubing assembly. For example, the sleeve element is configured to tightly hold (grip) the tubes, blocking the passage of liquids and/or air therethrough, e.g. via gaps between tubes.

In particular, the at least one outflow tube E4 and the plurality of the inflow tubes E1, E2, E2′, E3 are configured to be held together as a bundle, e.g. via the sleeve element 226, in a specific, packed arrangement.

In this way, the handling and positioning of the tubes is improved so the practitioner can identify and organize the tubing assembly reliably in a time-effective and easy manner. Advantageously, the sleeve element is 226 is designed to secure both the inflow tubes and at least one outflow tube together, ensuring the tubing assembly 220 passes smoothly through the incision in the body cavity.

In particular, the protective cover 226 comprises an outer wall defining a cavity (an inner volume or a free space) that is configured to surround (enclose) the multi-lumen tubing set including the plurality of inflow tubes and the at least one outflow tube.

For example, the plurality of inflow tubes and the at least one outflow tube are arranged within the protective cover 226 such that center points of two inflow tubes E2, E2′ and one outflow tube are disposed on a common line within the sleeve.

In particular, the at least one outflow tube E4 is disposed between the plurality of inflow tubes E1, and E2, E2′.

For example, the at least one outflow tube E4 is disposed in a central position, sandwiched between, at least, two of the inflow tubes.

In particular, the at least one outflow tube E4 is disposed between a twin-inflow tube E2, E2′ at one side and one other inflow tube E1 at other opposite side.

For example, the arrangement of the multi-lumen tubing set is such that two inflow tubes E2 and E2′ form the two extremities (two opposite sides) of the tubing assembly 220. In particular, these inflow tubes are configured to be placed on top of the heart (upper portion of the pericardial cavity).

In particular, the arrangement of the tubing set is further (designed) such that two of the plurality of inflow tubes E2 and E2′ are connected together, forming a twin-inflow tubes. More particularly, the twin-inflow tubes are configured to be placed on the bottom side of the hearth.

For example, one of the twin-inflow tubes is configured to carry the infusion fluid E2 and the other one the air E2′ (i.e. forming an air tube or a pressure measurement tube). In this way, the air channel is supported by the inflow tube to be kept clean. In other words, the flow of the infusion fluids prevents the undesirable blockage of the air tube by small clots or tissues.

In particular, the at least one outflow tube E4 has diameter larger than the plurality of inflow tubes.

In particular, the plurality of inflow tubes is of identical or different diameters.

For example, the at least one outflow tube has an outer diameter between 8 to 10 mm, preferably 9 mm and defines a lumen having an inner diameter of 5 to 7 mm, preferably 6 mm.

The plurality of inflow tubes, for example, each defines a lumen with an inner diameter of 1 to 3 mm, preferably 1.6 mm. Each of the inflow tubes has an outer diameter between 2 to 4 mm, preferably 3.2 mm.

In particular, the tubing assembly 220 further comprises a filler material that is configured to be disposed within the sleeve element to thereby fill-in the spaces available within the sleeve element 226. The unfilled areas within the sleeve element, not taken up by the inflow tubes and the outflow tube, are consequently filled with the filler material.

In this way, the gaps between the plurality of inflow tubes and the at least one outflow tube, which are disposed in the sleeve element, can be filled with the filler material, regardless of the shape or size of the tubes and the sleeve element.

Advantageously, by way of the sleeve element and the filler material disposed therein, it is possible to support the plurality of inflow tubes and the at least one outflow tube and hold them fix in place in a sealing manner. This, in turn, results in preventing undesired leakage (e.g. air or fluids) between the patient, e.g. the patient's body cavities, and the surrounding environment.

For example, a sleeve element is in the form of an extruded tube or molded sleeve that is configured to be fitted over the plurality of inflow tubes and the at least one outflow tube, over a certain length, at a proximal end of the tubing assembly, i.e. at a location proximate to the patient, in particular proximate to an incision location on the patient's body.

In this way, advantageously, the tubes exiting the patient's body are tightly hold together in a sealing manner.

For example, the tubing assembly 220 comprises three inflow tubes E1, E2 and E3 carrying infusion liquids into the patient (e.g. a body cavity), one pressure tube (air tube) E2′ carrying air, and one outflow tube E4 carrying effusion fluids from the patient.

In particular, the tubing assembly further comprises a cap 228 that is disposed at the distal end of the tubing assembly 220 (i.e. far from the patient, near to a flushing system).

The cap 228 is particularly arranged on the part of the tubing assembly 220 which extends from the patient's body, e.g. the pleural cavity or pericardial cavity, to the outside (i.e. it is disposed outside of the patient's body).

The cap assists in directing the tubing assembly 220 as it passes through the incision and leaves the body cavity.

The cap is configured to be removed from the tubing assembly 220 after disposing the proximal end 222 of the tubing assembly within the body cavity, to thereby allow the distal end 224 of the tubing assembly to be connected to the flushing system 100.

The tubing assembly 220 is configured to be connected to the tubing unit 134 of the flushing system 100, e.g. via the tubing connecter 160.

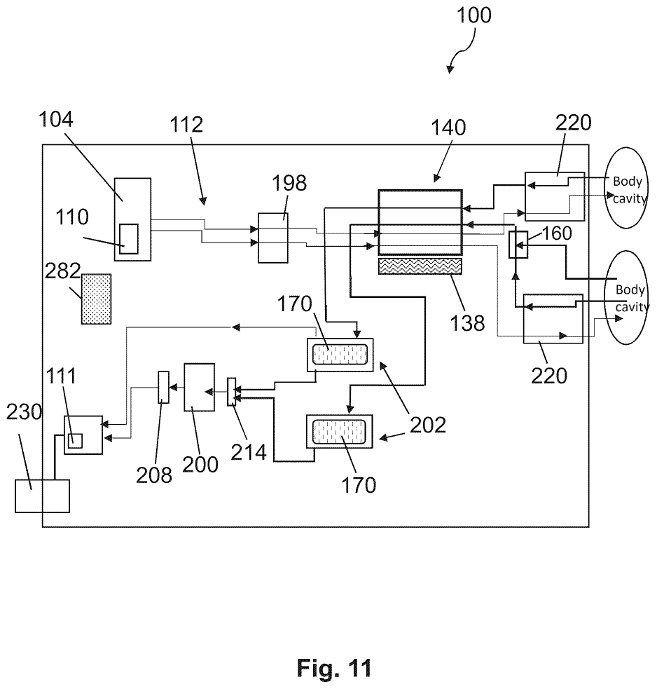

FIG. 11 schematically shows the flushing system 100.

In operation, the first pumping unit 198 is configured to pump infusion fluid from the inflow unit 104 towards the patient.

The first weighing unit 110 is configured to measure variations in the weight of the infusion fluid in the inflow unit (i.e. the infusion liquid container). This change in weight indicates, e.g. a decrease in the volume of infusion liquid in the infusion liquid container. By assessing the volume change of the infusion liquid over a specific duration, it becomes possible to ascertain the flow rate of infusion liquid directed toward the body cavity.

The infusion liquid flow rate of the first pump unit 198 is configured to be adjusted by the control unit 282, thereby a controlled volume of the infusion fluid is pumped to the pericardial cavity or a pleural cavity based on signals from multiple sensors.

The infusion fluid is configured to get heated while passing through the tubing interface unit 140 by the heater unit 138. Thereafter, the infusion fluid is introduced into the body cavity via the tube assembly 220 (i.e. through inflow lines of the tube assembly) that is connected to the patient.

The effusion liquid that is removed from the patient's body cavity (e.g. the pericardial cavity or a pleural cavity) through the tube assembly 220 (i.e. through outflow lines of the tube assembly) is guided to the respective intermediate buffer containers 202. The filtered effusion liquids are then separately pumped out of the intermediate buffer containers into the cuvette 210. The effusion fluid within the cuvette then is configured to be analyzed with the sensor unit disposed in the carrier unit 102.

One or more suction devices are arranged in the collection unit.

The suction devices are configured to create a relative low pressure, i.e. an under pressure of for example −15 mmHg, in the waste container. The reduction of the pressure may further be achieved by an ICU vacuum wall connector 230, thereby support the removal of the effusion fluids.

This relative low pressure can be used to draw effusion liquid from the pericardial cavity PC towards the effusion liquid container 202.

The second weighing unit 111 that is disposed under the collection unit 106 is configured to measure a volume/weight of effusion liquid in the waste container (e.g. effusion liquid container). On the basis of the change of volume/weight of effusion liquid over the course of time, the flow rate/volume of effusion liquid may be determined.

Any other sensor configured to determine the flow rate of effusion liquid from the pericardial cavity PC to the effusion liquid container 202 may also be used to calculate the flow rate/volume of effusion liquid. For example, the flow rate of effusion liquid may be determined by a flow rate sensor.

The flushing system according to the present invention serves a reliable and precise system that enables effective drainage and ensure patient safety. The quantity of fluids administered into to the patient's body and the quantity drained from the patient's body can be measured and monitored in real-time. More particularly, the removed effusion fluids from the body cavities can be separately filtered through intermediate containers and directed to an analysis chamber (e.g. a cuvette) of a sensor unit for distinct measurements and analysis. The intermediate containers include filters that are aligned in parallel to the direction of fluid flow.

The measurement unit according to the present invention is designed as an independent unit that is configured to be placed in a flushing system. The measurement unit is of a compact design comprising inflow and outflow pumping units, respectively, for delivering and extracting fluids. The measurement unit further equipped with two intermediate containers, each associated with a body cavity, for filtering the extracted fluids and for analysing the removed of fluids. Also, safety elements included in the measurement unit discharge the fluids from the intermediate fluid situations where the outflow pump(s) is(are) malfunctioning.

The tubing assembly according to the present invention has a smart design provided by one multi-lumen tube set. The multi-lumen tube set comprises a bundle of tubes securely joined together. The tubing assembly comprises inflow fluid tubes and outflow tubes that are configured to be coupled to a flushing system, facilitating the infusion of fluids and/or air and allow for the drainage of air or fluid while preventing their re-entry into the body cavity.

### REFERENCE SIGNS

100 a flushing system
102 a carrier unit
104 an inflow unit
106 a collection unit
108 an infusion fluid container
110 a first weighing unit
112 at least one inflow tube
114 a measurement unit
116 a user interaction unit
118 a mounting element
120 a housing of the carrier unit
122 a base portion
124 a top portion
126 an inner frame of the housing
128 a hollow chamber
130 a gripping element
132 a locking element
134 a tubing unit
136 guiding elements
138 a heater unit
140 a tubing interface unit
142 a base unit
144 a footplate
146 a casing
148 a base plate
150 a peripheral wall
152 a plurality of openings
156 an inner structure
157 a network of passages
158 a lid
160 a tubing connector
162 a pressure measurement tube
164 a waste container
166 a cover element
168 a handle
170
170 at least one filter element
172 a first coupling element
174 at least one second coupling element
175 an overflow protection valve
176 a first tubing section
178 a second tubing section
180 a third tubing section
182 a receiving element
184 a plurality of inflow tubes
186 a plurality of outflow tubes
188 a support element
190 a mounting surface
192 an upper edge
194 a lower edge
196 an entrance opening
198 a first pumping unit
200 sample ports
201 connector tubes
202 an intermediate fluid container
204 an inlet of the intermediate fluid container
206 a first outlet of the intermediate fluid container
208 a second outlet of the intermediate fluid container
210 a cuvette
212 a filter
214 a connector element
216 outflow tubes
218 a discharge tube
220 a tubing assembly
222 a proximal end
224 a distal end
226 a sleeve element
228 a cap
230 an intensive care unit (ICU) vacuum wall connector
232 a control unit

1. A flushing system for flushing a wound and/or a body cavity of a patient, the system comprising:
a carrier unit configured to be moved,
an inflow unit comprising at least one infusion fluid container containing infusion fluids, and
a collection unit configured to collect effusion fluids from the patient,
wherein the carrier unit is configured to accommodate the inflow unit and the collection unit,
wherein the inflow unit further comprises at least one first weighing unit for measuring a weight of the infusion fluids delivered to the patient, and
wherein the carrier unit comprises a second weighing unit for measuring a weight of the effusion fluids collected in the collection unit.

2. The flushing system according to claim 1,
wherein the flushing system further comprises a measurement unit configured to be operably connected to the inflow unit and the collection unit, and
wherein the measurement unit is configured to be disposed in an intermediate position, along a longitudinal axis of the carrier unit, between the inflow unit and the collection unit.

3. The flushing system according to claim 2,
wherein the measurement unit comprises two intermediate fluid containers for receiving the effusion fluids.

4. The flushing system according to claim 1,
wherein the flushing system further comprises a user interaction unit allowing a user to monitor flushing processes and/or results of analysis of the effusion fluids.

5. The flushing system according to claim 1,
wherein the inflow unit comprises at least one mounting element for holding the at least one first weighing unit and/or the at least one infusion fluid container.

6. The flushing system according to claim 1,
wherein the at least one first weighing unit is configured to retain the at least one infusion fluid container.

7. The flushing system according to claim 2,
wherein the carrier unit comprises a housing extending along a longitudinal axis from a bottom position to a top position, and
wherein the housing is configured to house the collection unit, the measurement unit, and the inflow unit therein, respectively, from the bottom position to the top position.

8. The flushing system according to claim 7,
wherein:
the housing comprises a pass-through chamber configured to accommodate the inflow unit therein, and/or
the flushing system further comprises a gripping element connected to the carrier unit.

9. The flushing system according to claim 2,
wherein the flushing system further comprises a tubing unit configured to carry the infusion fluids towards the patient, and to carry the effusion fluids from the patient towards the flushing system,
wherein the tubing unit is configured to be connected to the measurement unit and the patient.

10. The flushing system according to claim 1,
wherein the collection unit further comprises a waste container for collecting the effusion fluids from the patient, and/or a cover element that is configured to be connected to the waste container.

11. The flushing system according to claim 1,
wherein the flushing system further comprises guiding elements configured to guide positioning of the tubing unit towards the carrier unit.

12. The flushing system according to claim 9,
wherein the flushing system further comprises a tubing interface unit configured to retain, at least partly, the tubing unit providing a fluid communication between the carrier unit and the patient.

13. The flushing system according to claim 1,
wherein the flushing system further comprises at least one tubing connector configured to combine streams of the effusion fluids received from the patient.

14. The flushing system according to claim 13,
wherein the at least one tubing connector is configured to fluidly connect an outflow tube included in a tubing assembly, attached to the patient, with a further outflow tube attached to the patient, and
wherein the tubing assembly and the further outflow tube are both connected to a common body cavity.

15. The flushing system according to claim 9,
wherein the flushing system further comprises a heater unit configured to heat at least a section of the tubing unit providing a fluid communication between the carrier unit and the patient.

16. The flushing system according to claim 1, wherein the body cavity is the pericardial cavity and/or the pleural cavity of the patient.

17. The flushing system according to claim 2, wherein the measurement unit comprises at least one analysis chamber and/or at least one pumping unit.

18. The flushing system according to claim 3, wherein the two intermediate fluid containers for receiving the effusion fluids comprise a first intermediate fluid container configured to receive the effusion fluids from a pleural cavity and a second intermediate fluid container configured to receive the effusion fluids from a pericardial cavity.

19. The flushing system according to claim 6, wherein the at least one first weighing unit is configured to be connected to the carrier unit via a shock mounting.

20. The flushing system according to claim 8, wherein the gripping element is connected to the carrier unit at a position to provide for a protection for the inflow unit, and wherein the gripping element is configured to protect the at least one first weighing unit and/or the at least one infusion fluid container.
